(19) United States
(12) Patent Application Publication
(10) Pub. No.: US 2026/0144463 A1
(43) Pub. Date: May 28, 2026

(54) CALCULATION OF GASEOUS BIOMARKER CONCENTRATION IN TISSUE VIA AN INVERSE BOUNDARY PROBLEM

(71) Applicant: The General Hospital Corporation, Boston, MA (US)

(72) Inventors: Juan Cascales Sandoval, Boston, MA (US); Conor L. Evans, Boston, MA (US)

(21) Appl. No.: 19/123,035

(22) PCT Filed: Nov. 29, 2023
(86) PCT No.: PCT/US2023/081512

§ 371 Date: Apr. 21, 2025

Related U.S. Application Data
(60) Provisional application No. 63/429,074, filed on Nov. 30, 2022.
(60) Provisional application No. 63/490,982, filed on Mar. 17, 2023.

(51) Int. Cl. A61B 5/1455

(52) U.S. Cl. CPC A61B 5/14551

(57) ABSTRACT

Systems and methods for determining a level of a parameter of an analyte: cause a photon source to direct photons at a probe sensitive to the analyte and excite the probe to emit light; receive optical data from a photodetector based on the light emitted from the probe; determine, based on optical data from the photodetector, a difference between a first level of a parameter of the analyte at a first location adjacent a first surface of the probe and an intermediate level of the parameter of the analyte at an intermediate location between the first location and a second location adjacent a second surface of the probe; and determine a second level of the parameter of the analyte adjacent the second surface of the probe based on the difference between the first level of the parameter of the analyte and the intermediate level of the parameter of the analyte.

### CROSS-REFERENCE TO RELATED APPLICATIONS

This application is based on, claims benefit of, and claims priority to U.S. Patent Application No. 63/429,074 filed on Nov. 30, 2022, and U.S. Patent Application No. 63/490,982 filed on Mar. 17, 2023, which are hereby incorporated by reference herein in their entirety for all purposes.

### STATEMENT REGARDING FEDERALLY SPONSORED RESEARCH

This invention was made with government support under grant number FA9550-17-1-0277 awarded by the Air Force Office of Scientific Research and grant number HU0001-17-2-0009 awarded by the Henry M. Jackson Foundation for the Advancement of Military Medicine. The government has certain rights in the invention

### BACKGROUND

### 1. Field of the Invention

The invention relates to sensor systems and methods for detecting a concentration of an analyte, and more particularly to transcutaneous gas concentration monitor devices that provide accurate tissue measurement of gases, such as O₂.

### 2. Description of the Related Art

The quantitative assessment of gas (e.g., O₂, CO₂) concentration in living tissue is critical in diagnosing and treating various conditions, especially in situations where conventional clinical tools prove inadequate, such as in the absence of blood flow or when blood pooling occurs [Ref. 1]. To directly measure the gases available to tissue noninvasively, one can use transcutaneous oxygen tension monitor (TCOM) devices. The measurement of transcutaneous oxygenation (transcutaneous pO₂ or tcpO₂) can be a valuable metric in burns, the application of tourniquets and used to detect the onset of ischemia in diabetic ulcers, among other scenarios [Ref. 2-5]. However, the high cost, large size, and cumbersome operation of currently available transcutaneous measurement equipment pose significant obstacles to the practical utility of this technology. Furthermore, these bulky devices require relatively pristine conditions to provide reliable measurements, such as the need for patients to remain immobile, the need for long equilibration times, and in the case of transcutaneous oxygen monitors (TCOM), the requirement to heat skin to temperatures over 40° C.

Of these challenges, an important limitation of transcutaneous gas measurements is the question of whether the measurements reflect the true partial pressure (e.g., pO₂) of tissue on the skin surface [Ref. 6], as well as the need for long equilibration periods in order to obtain stable readings. This arises from the fact that these devices sit not within, but atop the skin; values measured by the devices can be delayed from that of skin due to time required for oxygen to diffuse into and equilibrate with the headspace of the device. Upon application of these devices, an exponential drop is typically observed from atmospheric; (for pO₂, this starts close to 160 mmHg and drops to a value between 60 and 100 mmHg). The speed and amplitude of the decay depends on the volume of trapped air between the detectors and skin, the diffusion of the gas through the different materials used, skin gas consumption, and other factors [Ref. 7, 8]. If materials impermeable to gases are used as a barrier against atmospheric gases [Ref. 9], it has been assumed that, given enough time, the obtained concentration readings correspond to the real tissue value. However, for applications in which the sensors require long-term wear, such as in postoperative scenarios [Ref. 10], the sensors should ideally make use of semibreathable materials in order to allow the skin to “breathe” (i.e., partially allow diffusion of moisture, oxygen, etc.), which means the equilibrium value achieved is an intermediate value between atmosphere and skin gas concentrations. Additionally, it is conceivable for some applications to require avoiding direct contact between skin and gas sensing materials, e.g., using sensing materials that could be potentially harmful [Ref. 11], through the addition of semipermeable barrier layers, further altering the differences between the measured and the true tissue values.

Therefore, what is needed is transcutaneous gas tension monitor devices that systematically produce true tissue tension readings, while reducing the need for long equilibration times, despite the use of barrier or intermediate layers between tissue and sensors.

### SUMMARY OF THE DISCLOSURE

To address these limitations, the present disclosure provides systems and methods to address this limitation such that transcutaneous gas tension monitor device measured gas concentrations can be translated into true gas tension values while employing materials which can be used for long term wear applications.

In one aspect, the present disclosure provides a sensor system comprising: a probe sensitive to an analyte, the probe having a body including a first surface and an opposite second surface; a photon source configured to direct photons at the probe, the probe emitting light in response to receiving photons from the photon source; a photodetector configured to detect the light emitted from the probe; and a controller in communication with the photon source and the photodetector. The controller can be configured to execute a program stored in the controller to: (i) cause the photon source to direct photons at the probe and excite the probe to emit light in response to receiving the photons, (ii) receive optical data from the photodetector based on the interaction between the light emitted from the probe and the photodetector, (iii) determine a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and (iv) determine a second level of the parameter of the analyte adjacent the second surface of the probe based on the difference between the first level of the parameter of the analyte and the intermediate level of the parameter of the analyte.

In one embodiment of the sensor system, the controller executes the program stored in the controller to determine the second level of the analyte adjacent the second surface of the probe by solving a diffusion mathematical problem including the first level of the analyte and the intermediate level of the analyte.

In one embodiment of the sensor system, the controller executes the program stored in the controller to determine the second level of the analyte adjacent the second surface of the probe by solving an inverse boundary condition mathematical problem including the first level of the analyte and the intermediate level of the analyte. In one embodiment of the sensor system, the inverse boundary condition mathematical problem includes a thickness dependent diffusivity variable. In one embodiment of the sensor system, the inverse boundary condition mathematical problem includes Dirichlet boundary conditions.

In yet another aspect, the present disclosure provides a sensor system comprising: a probe sensitive to an analyte, the probe having a body including a first surface and an opposite second surface; a photon source configured to direct photons at the probe, the probe emitting light in response to receiving photons from the photon source; a photodetector configured to detect the light emitted from the probe; and a controller in communication with the photon source and the photodetector. The controller can be configured to execute a program stored in the controller to: (i) cause the photon source to direct photons at the probe and excite the probe to emit light in response to receiving the photons, (ii) receive optical data from the photodetector based on the interaction between the light emitted from the probe and the photodetector, (iii) determine, based on the optical data from the photodetector, a gradient of a parameter of the analyte extending from a first location adjacent the first surface of the probe to an intermediate location between the first location and a second location adjacent the second surface of the probe, and (iv) calculate, based on the gradient, a level of the parameter of the analyte adjacent the second surface of the probe.

In yet another aspect, the present disclosure provides a method for determining a level of a parameter of an analyte. The method can comprise: (a) positioning a probe proximate to a surface to monitor an analyte; (b) positioning a photon source to deliver photons to the probe to excite the probe; (c) positioning a photodetector to receive light emitted by the probe in response to being emitted by the photon source; (d) causing the photon source to direct photons at the probe and excite the probe to emit light in response to receiving the photons; (e) collecting optical data from the photodetector based on the interaction between the light emitted from the probe and the photodetector; (f) determining a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and (g) determining a second level of the parameter of the analyte adjacent the second surface of the probe based on the difference between the first level of the parameter of the analyte and the intermediate level of the parameter of the analyte. In one embodiment of the method, step (g) comprises determining the second level of the analyte adjacent the second surface of the probe by solving a diffusion mathematical problem including the first level of the analyte and the intermediate level of the analyte. In one embodiment of the method, step (g) comprises determining the second level of the analyte adjacent the second surface of the probe by solving an inverse boundary condition mathematical problem including the first level of the analyte and the intermediate level of the analyte. The inverse boundary condition mathematical problem can include a thickness dependent diffusivity variable. The inverse boundary condition mathematical problem can include Dirichlet boundary conditions.

In yet another aspect, the present disclosure provides a method for determining a level of a parameter of an analyte. The method can comprise: (a) positioning a probe proximate to a surface to monitor an analyte; (b) positioning a photon source to deliver photons to the probe to excite the probe; (c) positioning a photodetector to receive light emitted by the probe in response to being emitted by the photon source; (d) causing the photon source to direct photons at the probe and excite the probe to emit light in response to receiving the photons; (e) collecting optical data from the photodetector based on the interaction between the light emitted from the probe and the photodetector; (f) determining, based on the optical data from the photodetector, a gradient of a parameter of the analyte extending from a first location adjacent the first surface of the probe to an intermediate location between the first location and a second location adjacent the second surface of the probe; and (g) calculating, based on the gradient, a level of the parameter of the analyte adjacent the second surface of the probe.

In still another aspect, the present disclosure provides a computer system comprising instructions stored on a non-transitory computer readable medium to cause at least one processor on a computer to: (i) cause a photon source to direct photons at a probe sensitive to an analyte and excite the probe to emit light in response to receiving the photons, the probe having a body including a first surface and an opposite second surface; (ii) receive optical data from a photodetector based on the interaction between the light emitted from the probe and the photodetector; (iii) determine a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and (iv) determine a second level of the parameter of the analyte adjacent the second surface of the probe based on the difference between the first level of the parameter of the analyte and the intermediate level of the parameter of the analyte. In one embodiment of the computer system, the at least one processor determines the second level of the analyte adjacent the second surface of the probe by solving a diffusion mathematical problem including the first level of the analyte and the intermediate level of the analyte. In one embodiment of the computer system, the at least one processor determines the second level of the analyte adjacent the second surface of the probe by solving an inverse boundary condition mathematical problem including the first level of the analyte and the intermediate level of the analyte. The inverse boundary condition mathematical problem can include a thickness dependent diffusivity variable. The inverse boundary condition mathematical problem can include Dirichlet boundary conditions.

In yet another aspect, the present disclosure provides a computer system comprising instructions stored on a non-transitory computer readable medium to cause at least one processor on a computer to: (i) cause a photon source to direct photons at a probe sensitive to an analyte and excite the probe to emit light in response to receiving the photons, the probe having a body including a first surface and an opposite second surface; (ii) receive optical data from a photodetector based on the interaction between the light emitted from the probe and the photodetector; (iii) determine, based on the optical data from the photodetector, a gradient of a parameter of the analyte extending from a first location adjacent the first surface of the probe to an intermediate location between the first location and a second location adjacent the second surface of the probe, and (iv) calculate, based on the gradient, a level of the parameter of the analyte adjacent the second surface of the probe.

One advantage of systems and methods according to the present disclosure is that the systems and methods report true tissue gas concentration by taking into account the gas's concentration gradient in a transcutaneous tension monitor when calculating true gas concentration on the skin surface.

Another advantage of systems and methods according to the present disclosure is that the systems and methods dramatically reduce the equilibration time, from around 30-60 minutes in conventional devices to a few minutes. Commercial devices and methods typically require equilibration periods of around 30-60 minutes which interferes with measurements needed in clinic and medical settings, in particular emergency medicine applications, where knowledge of tissue gas concentrations (e.g., pO₂) is required in seconds to minutes.

Sensor systems and methods according to the present disclosure have many applications. Non-limiting examples include: (i) software which can be adapted for any transcutaneous sensors to obtain skin surface readings. (ii) algorithms which can be included as part of the signal-processing, (iii) calibration tools that can be used to determine material properties for the software and algorithms. These products can find use in many areas of analyte measurement, including commercial transcutaneous sensors that measure true skin O₂ and CO₂, based on wearable technology such as that developed by Massachusetts General Hospital, Wellman Center for Photomedicine, Boston, Massachusetts, USA.

Beyond gas sensing, systems and methods according to the present disclosure can find use in other biomedical applications, including sensing liquids and liquid-dissolved compounds including drugs and metabolites in tissues. While there are differences between gas and liquid diffusion, the diffusion process is similar and thus the application described herein applies. There are additional applications in materials science and materials characterization where there are dynamic diffusion processes. These include: the development, testing, and functional monitoring of batteries, including lithium Ion and liquid-type batteries; and manufacturing of materials including polymers, carbon-fiber materials and composites, and multilayered materials. For example, carbon-fiber materials must be manufactured in the absence of oxygen, which otherwise would make the final material brittle. Sensors for gases, such as systems according to the present disclosure, can detect the true material gas concentrations which can be of significant aid in this critical manufacturing step.

These and other features, aspects and advantages of various embodiments of the present disclosure will become better understood with regard to the following description, appended claims, and accompanying Figures.

### BRIEF DESCRIPTION OF THE DRAWINGS

FIG. 1A shows a wearable transcutaneous oxygenation monitor system according to the present disclosure in which a numerical model based on an inverse boundary problem of a parabolic equation with Dirichlet boundary conditions is used to obtain accurate tissue pO₂ measurements without the need for long equilibration times.

FIG. 1B shows: in Panel (a), a layer structure of the oxygen-sensing film, and it is clinical application together with the wearable oxygen sensor; and in Panel (b), a schematic diagram of the oxygen gradient through the different layers of the oxygen sensing film, showing the unknown true tissue pO₂ (RBC=???).

FIG. 2 shows testing of the inverse boundary problem algorithm with sample problems with Panel (a) Neumann and Panel (b) Dirichlet boundary conditions. The right boundary condition (RBC) and left boundary condition (LBC) are initially known, and the direct problem for an intermediate value of x=x_{*} is calculated with a direct diffusion model. Then, the inverse boundary problem is used to calculate the RBC (RBC_{IBP}) using u(x_{*}, t), which closely matches the initially known values.

FIG. 3 shows in Panel (a), the film structure is described in cylindrical coordinates to reduce the diffusion problem to one dimension, as ø_{PPMA}>>d_{PPMA}; in Panel (b), the system to perform a control experiment; in Panel (c), the fitting of the inverse boundary problem algorithm to the measured right boundary, using the known left boundary and intermediate u(x_{*}, t)=pO₂^{TCOM}, from which x_{*} and the diffusion coefficients of the film layers are found, wherein the insets show underestimation in the change of pO₂ of the raw TCOM reading during the “occlusion” period; and in Panel (d), the residual of the fit, showing the small difference between fit and signal.

FIG. 4 shows: in Panel (a), the direct diffusion model with Dirichlet conditions is successful in reconstructing the value of the intermediate value from the pO₂ readings of the wearable device, matching well the experimental data, using the fitting constants from the inverse model fit, and the constant left boundary of 160 mmHg and measured right boundary (RBC_{IBP}); and in Panel (b), the oxygen profile at different time points during the measurement (left axis) and diffusion coefficient profile (right axis) found from the fit.

FIG. 5 shows the fitting of the inverse boundary problem (Panels a, c, e, g) and residual (Panels b, d, f, h) of measurements performed on the four additional films.

FIG. 6 shows: in Panel (a), application of the inverse boundary problem algorithm to TCOM clinical data, where blood flow to the lower leg was restricted for 10 minutes (highlighted region) wherein the recalculated pO₂ presents values closer to those of tissue; and in Panel (b), the relative change of pO₂ during the occlusion, with the true tissue pO₂ showing a larger and sharper change compared to the raw TCOM reading.

Like reference numerals will be used to refer to like parts from Figure to Figure in the following detailed description. Any drawings herein are not shown to scale. Where dimensions are given in the text or figures, these dimensions are merely example values which could be used with one or more example implementations and do not limit the scope of the disclosed invention. Some embodiments of the disclosure are described herein with reference to the accompanying figures. The description, together with the figure, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

### DETAILED DESCRIPTION

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It will be appreciated by those skilled in the art that while the disclosed subject matter is described herein in connection with particular embodiments and examples, the invention is not necessarily so limited, and that numerous other embodiments, examples, uses, modifications and departures from the embodiments, examples and uses are intended to be encompassed by the claims attached hereto.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise.

In non-limiting embodiments, this invention describes mathematical methods and calibration methods to obtain an estimate of the concentration of a gaseous biomarker in tissue from a non-invasive measurement on the skin surface. While the method includes an embodiment of obtaining oxygen concentration in tissue and describes a specific embodiment of oxygen as the biomarker of interest, the method of the invention can be applied to other gaseous compounds emanating from skin, such as carbon dioxide, nitric oxide, or volatile organic compounds. A method of the invention can further be applied to any scenario where the sensing of an analyte in a material is required, but the sensor exists outside the material itself or is separated from the material by a semi-permeable barrier. Thus, this approach has applications in biomedicine, but also likely in material characterization, fabrication, and manufacturing.

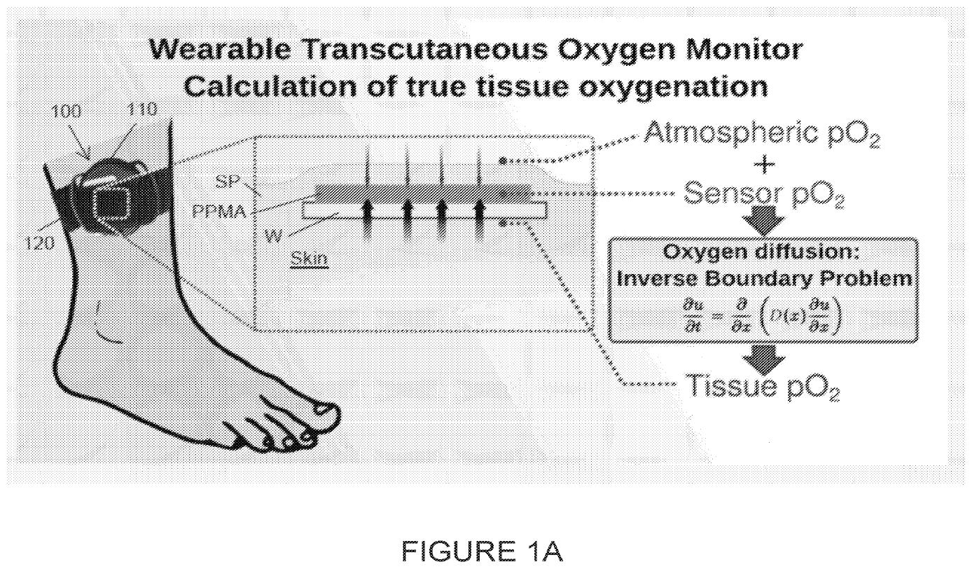

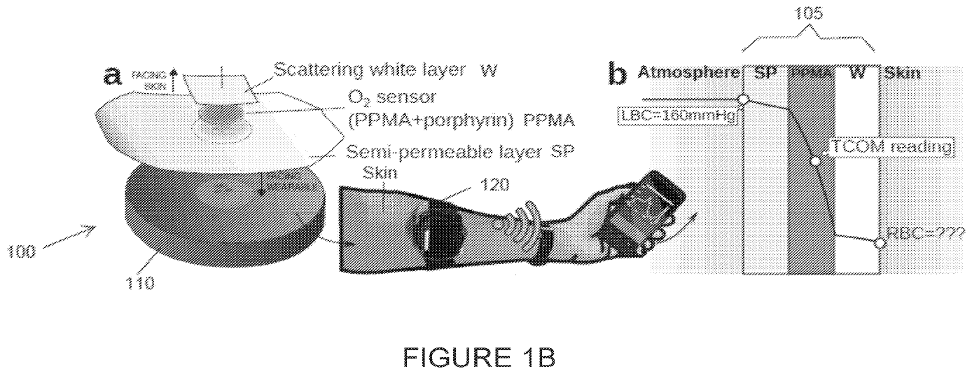

Referring now to FIG. 1B, there is shown one non-limiting example embodiment of a wearable device 100 according to the invention for transcutaneous oxygen monitoring. The device 100 includes a photoluminescent oxygen-sensitive probe comprising a film 105 including a polymer matrix and a porphyrin molecule. A photon source is configured to direct photons at a wavelength at the probe. A photodetector is configured to detect light emitted from the probe when the photon source directs photons at the probe. Illumination from the photon sources can be modulated (e.g., square, sinusoidal, triangular, sawtooth, etc.) or constant. The photodetector can be a PIN photodiode. The device 100 includes electronics 110 having a controller in electrical communication with the photon source and the photodetector. The controller is configured to execute a program stored in the controller to calculate a level of oxygen adjacent the probe from an electrical signal received from the photodetector. The device 100 includes a highly breathable white scattering layer W, and a transparent semipermeable transparent membrane SP partially occluding the skin from atmospheric oxygen. Since the device 100 can directly measure pO₂, it does not require perfusion or presence of underlying blood vessels. The optical device 100 can require low preparation time, and a readout of the device 100 can be essentially instantaneous. With the optical device 100, bedside calibration and heating may not be required, underlying blood flow may not be needed, and minimal equilibration time before getting results that can be presented on a simple readout, thereby providing a solution to the problems that plague clinical oxygen sensing as a whole.

In the device 100, the photoluminescent oxygen-sensitive probe can comprise a polymer matrix. In some embodiments, the polymer matrix comprises a hydrophobic polymer. In some embodiments, the polymer matrix comprises a polymer selected from the group consisting of acrylate polymers and methacrylate polymers and blends and copolymers thereof. Non-limiting example acrylate polymers include poly(acrylic acid), poly(methyl acrylate), poly(ethyl acrylate), poly(propyl acrylate), and poly(butyl acrylate). Non-limiting example methacrylate polymers include poly(methyl methacrylate), poly(ethyl methacrylate), poly(propyl methacrylate), poly(butyl methacrylate), and poly(hydroxyethyl methacrylate). In some embodiments, the polymer matrix comprises a polymer selected from the group consisting of alkyl methacrylate polymers. In some embodiments, the polymer matrix comprises poly(propyl methacrylate) (PPMA). In some embodiments, the polymer matrix comprises a polymer selected from the group consisting of siloxane polymers and blends and copolymers thereof. In a non-limiting example, the polymer matrix comprises polydimethylsiloxane (PDMS). For instance, silicones such as PDMS can be extremely gas-permeable and can allow rapid readout of tissue gas concentration dynamics.

In a non-limiting example, the porphyrin molecule in the photoluminescent oxygen-sensitive probe can be a metalloporphyrin that can emit red phosphorescence when excited by blue light, and the phosphorescence intensity and lifetime can be inversely proportional to pO₂. A reference sensor in the form of a green-emitting dye can also be incorporated into the photoluminescent oxygen-sensitive probe to serve as a reference standard for precise pO₂ measurements. In another non-limiting example, a fluorophore sensitive to the presence of CO₂ bound to a material can be used detect the concentration of CO₂.

In some embodiments, porphyrin-based, oxygen sensing molecules embedded in the polymeric matrix are designed to provide extremely high sensitivity and accuracy for the measurement of tissue oxygenation. Porphyrin-based, oxygen sensing molecules can be built via a modular synthetic pathway that enables the tailoring of both the oxygen sensing molecules' oxygen sensitivity range and the oxygen sensing molecules' compatibility with the matrix material that the oxygen sensing molecules can be embedded in. The matrix material can further be configured to tailor the oxygen sensing molecules' oxygen sensitivity range. In one embodiment, the change in the film 105 includes a change in the phosphorescence. For example, the oxygen sensing molecules can be specifically designed to feature bright, red phosphorescence emission, with a visual response to changes in oxygenation level that can be seen under ambient light. These properties simplify the collection and interpretation of their oxygen-dependent emission, enabling the analysis to be performed with simple and inexpensive equipment

In one embodiment, the oxygen-sensing molecule comprises a phosphorescent meso-unsubstituted porphyrin having the Formula (I):

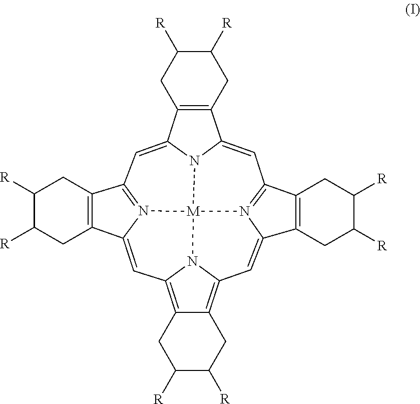

wherein M is a metal, wherein each R is independently an atom or a group of atoms, and wherein at least one R is —OR¹, wherein R¹ is an atom or a group of atoms.

In the porphyrin of Formula (I), R¹ may be selected from the group consisting of hydrogen, substituted or unsubstituted alkyl, substituted or unsubstituted alkyl carbonyl, substituted or unsubstituted alkenyl, substituted or unsubstituted alkynyl, substituted or unsubstituted cycloalkyl, substituted or unsubstituted heterocycloalkyl, substituted or unsubstituted aryl, heteroaryl, halo, cyano, and nitro. In one example of the porphyrin of Formula (I), R¹ is hydrogen. In another example of the porphyrin of Formula (I), R¹ is alkynyl, such as 2-propynyl (propargyl). In yet another example of the porphyrin of Formula (I), R¹ is alkyl carbonyl, such as 2,2-dimethylpropanoyl (also known as trimethylacetyl or pivaloyl). In the porphyrin of Formula (I), a plurality of R can be —OR¹, and optionally, every R can be —OR¹.

In one example of the porphyrin of Formula (I), R¹ includes a triazolyl group. The triazolyl group may be bonded to O via an alkyl chain. In one example of the porphyrin of Formula (I), R¹ includes an alkylglutamate group. R¹ may terminate in a pair of alkylglutamate groups. In another example of the porphyrin of Formula (I), R¹ includes a triazolyl group, and R¹ terminates in a pair of ethylglutamate groups, and every R is —OR¹. In one example of the porphyrin of Formula (I), the metal is platinum or palladium.

The porphyrin of Formula (I) may be an oxygen-sensitive phosphor whose emission intensity is dependent on oxygen partial pressure. In one example of the porphyrin of Formula (I), the porphyrin can be excited when illuminated at a first wavelength in a range of 350-600 nanometers, followed by emission of phosphorescence at a second wavelength in a range of 600-700 nanometers. The first wavelength can be 532 nanometers, and the second wavelength can be 644 nanometers. The first wavelength can also be 546 nanometers and the second wavelength can be 674 nanometers.

In another embodiment, the oxygen-sensing molecule comprises a phosphorescent meso-unsubstituted porphyrin having the Formula (II):

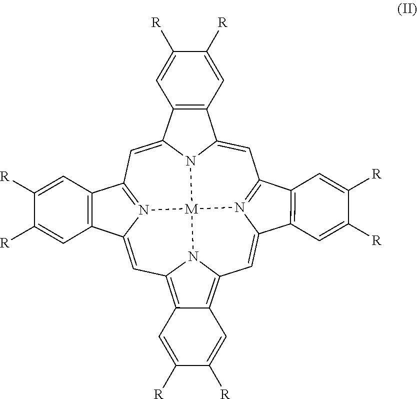

wherein M is a metal, wherein each R is independently an atom or a group of atoms, and wherein at least one R is —OR¹, wherein R¹ is an atom or a group of atoms.

In the porphyrin of Formula (II), R¹ may be selected from the group consisting of hydrogen, substituted or unsubstituted alkyl, substituted or unsubstituted alkyl carbonyl, substituted or unsubstituted alkenyl, substituted or unsubstituted alkynyl, substituted or unsubstituted cycloalkyl, substituted or unsubstituted heterocycloalkyl, substituted or unsubstituted aryl, heteroaryl, halo, cyano, and nitro. In one example of the porphyrin of Formula (II), R¹ is hydrogen. In another example of the porphyrin of Formula (II), R¹ is alkynyl, such as 2-propynyl (propargyl). In yet another example of the porphyrin of Formula (II), R¹ is alkyl carbonyl, such as 2,2-dimethylpropanoyl (also known as trimethylacetyl or pivaloyl). In the porphyrin of Formula (II), a plurality of R can be —OR¹, and optionally, every R can be —OR¹.

In one example of the porphyrin of Formula (II), R¹ includes a triazolyl group. The triazolyl group may be bonded to O via an alkyl chain. In one example of the porphyrin of Formula (II), R¹ includes an alkylglutamate group. R¹ may terminate in a pair of alkylglutamate groups. In another example of the porphyrin of Formula (II), R¹ includes a triazolyl group, and R¹ terminates in a pair of ethylglutamate groups, and every R is —OR¹. In one example of the porphyrin of Formula (II), the metal is platinum or palladium.

The porphyrin of Formula (II) may be an oxygen-sensitive phosphor whose emission intensity is dependent on oxygen partial pressure. In one example of the porphyrin of Formula (II), the porphyrin can be excited when illuminated at a first wavelength in a range of 350-650 nanometers, followed by emission of phosphorescence at a second wavelength in a range of 700-800 nanometers. The first wavelength can be 594 nanometers, and the second wavelength can be 740 nanometers. The first wavelength can be 605 nanometers, and the second wavelength can be 770 nanometers. The first wavelength can also be 600-615 nanometers and the second wavelength can be 760-800 nanometers. In one non-limiting embodiment of the device 100, the photon source can be a light-emitting diode emitting light having a wavelength of 405 nanometers. The photons from the photon source can be filtered using a long-pass filter.

The controller can be a microcontroller, or system-on-a-chip, and can comprise a memory which can be a non-transitory memory that can store executable programs on the controller. In some embodiments, the controller can store an analyte (e.g., oxygen) calculation program that can calculate a level of analyte (e.g., oxygen) adjacent the probe from one or more electrical signals received from the photodetector. The controller can also include an output, which can be a wire bundle. The output can connect to an external interface which can be used for at least one of displaying, storing, and analyzing the results of the executable program of the device 100. The device 100 may include a display in electrical communication with the controller, wherein the controller is configured to execute the program stored in the controller to display the level of oxygen on the display and/or store values of the level of oxygen at a plurality of times over a time period. In other embodiments, the controller can be configured to have a wireless output; the wireless output can perform wireless communication. Non-limiting examples of wireless communication that can be incorporated are Wi-Fi, Bluetooth®, near-field communication, cellular network, radiofrequency, etc. The controller can comprise an onboard power source, for example a battery, which can provide power to the controller such that the photon source(s), the photodetector(s), and the controller can be powered. In other embodiments, the controller can comprise an external power source, for example electrical connection to grid power, such that the photon source(s), the photodetector(s), and the controller can be powered.

In the device 100, the highly breathable white scattering layer W can comprise a silicone film including a pigment. The pigment can be white. The scattering layer W allows for the phosphorescence emission to be backscattered to the photodetector. The scattering layer W also serves as an optical insulation, preventing external lighting from affecting the measurement, yielding a reading that is independent of tissue color or skin tone. In the device 10, the semipermeable transparent membrane SP can partially occlude the tissue from atmospheric oxygen to seal out room air from the probe and scattering layer W, allowing the material to equilibrate to tissue pO₂. The device 100 for transcutaneous O₂ sensing is biocompatible since only the multilayer film makes contact with the skin. The probe is prevented from direct contact with the tissue via the light scattering layer W.

Now that the components of the device 100 have been described in detail, the method of operating the optical device 100 can be understood. While the device 100 is especially useful in detecting a concentration of oxygen, the device 100 can be used in a method for detecting a concentration of other analytes. In a non-limiting example method of operating the optical device 100, the photon source is activated by the controller to direct photons at a wavelength at the probe. The photodetector detects light emitted from the probe when the photon source directs photons at the probe. The controller executes a program stored in the controller to calculate a level of a parameter of the analyte adjacent the probe based on the light emitted from the probe detected by the photodetector.

The controller can execute the program stored in the controller to calculate the level of the parameter of the analyte adjacent the probe based on an algorithm, calibration, and/or normalization factor determined by directing photons at the wavelength at the probe to account for variations in brightness of the polymer matrix. The controller can execute the program stored in the controller to calculate the level of the parameter of the analyte adjacent the probe based on a luminescence (e.g., phosphorescence) ratio providing a metric that is proportional to the level of the parameter of the analyte adjacent the probe and is normalized using the normalization factor. The controller can execute the program stored in the controller to calculate the level of the parameter of the analyte adjacent the probe based on a calibration algorithm such that the device can report the level of the parameter of the analyte adjacent the probe adjacent the probe via wired or wireless communication (e.g., USB or Wi-Fi). The calibration algorithm may use different calibration parameters depending on whether a derivative of the luminescence (e.g., phosphorescence) ratio is positive or negative.

In one aspect, the present invention provides a sensor system comprising: a probe sensitive to an analyte, the probe having a body including a first surface and an opposite second surface; a photon source configured to direct photons at the probe, the probe emitting light in response to receiving photons from the photon source; a photodetector configured to detect the light emitted from the probe; and a controller in communication with the photon source and the photodetector. The controller can be configured to execute a program stored in the controller to: (i) cause the photon source to direct photons at the probe and excite the probe to emit light in response to receiving the photons, (ii) receive optical data from the photodetector based on the interaction between the light emitted from the probe and the photodetector, (iii) determine a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and (iv) determine a second level of the parameter of the analyte adjacent the second surface of the probe based on the difference between the first level of the parameter of the analyte and the intermediate level of the parameter of the analyte.

In one embodiment of the sensor system, the controller executes the program stored in the controller to determine the second level of the analyte adjacent the second surface of the probe by solving a diffusion mathematical problem including the first level of the analyte and the intermediate level of the analyte.

In one embodiment of the sensor system, the controller executes the program stored in the controller to determine the second level of the analyte adjacent the second surface of the probe by solving an inverse boundary condition mathematical problem including the first level of the analyte and the intermediate level of the analyte. In one embodiment of the sensor system, the inverse boundary condition mathematical problem includes a thickness dependent diffusivity variable. In one embodiment of the sensor system, the inverse boundary condition mathematical problem includes Dirichlet boundary conditions.

In yet another aspect, the present invention provides a sensor system comprising: a probe sensitive to an analyte, the probe having a body including a first surface and an opposite second surface; a photon source configured to direct photons at the probe, the probe emitting light in response to receiving photons from the photon source; a photodetector configured to detect the light emitted from the probe; and a controller in communication with the photon source and the photodetector. The controller can be configured to execute a program stored in the controller to: (i) cause the photon source to direct photons at the probe and excite the probe to emit light in response to receiving the photons, (ii) receive optical data from the photodetector based on the interaction between the light emitted from the probe and the photodetector, (iii) determine, based on the optical data from the photodetector, a gradient of a parameter of the analyte extending from a first location adjacent the first surface of the probe to an intermediate location between the first location and a second location adjacent the second surface of the probe, and (iv) calculate, based on the gradient, a level of the parameter of the analyte adjacent the second surface of the probe.

In one embodiment of the sensor system, the parameter is partial pressure. In one embodiment of the sensor system, the analyte includes at least one of oxygen, carbon dioxide, nitric oxide, and volatile organic compounds. In one embodiment of the sensor system, the analyte includes oxygen.

In one embodiment of the sensor system, a section of the sensor system is adapted to define a zone adjacent a third surface when the section of the sensor system is positioned over a portion of the third surface, and the probe is in fluid communication with the zone. In one embodiment of the sensor system, the third surface comprises skin of a patient, and the analyte includes transcutaneous oxygen.

In one embodiment of the sensor system, the probe comprises a layer of a polymeric material embedded with a metalloporphyrin. In one embodiment of the sensor system, an oxygen permeable scattering layer is positioned between the layer of the polymeric material and the third surface. The gas permeable scattering layer increases collected phosphorescence signal reaching the photodetector and serves as optical insulation.

In one embodiment of the sensor system, a semi-permeable layer positioned between the layer of the polymeric material and the photodetector. In one embodiment of the sensor system, the semi-permeable layer is semi-permeable to oxygen diffusion therethrough.

In one embodiment of the sensor system, the third surface comprises skin of a patient, the analyte includes transcutaneous oxygen, and an equilibration time of oxygen in the zone is less than 30 minutes when the section of the sensor system is positioned over the portion of the skin, or less than 20 minutes when the section of the sensor system is positioned over the portion of the skin, or less than 10 minutes when the section of the sensor system is positioned over the portion of the skin.

In one embodiment of the sensor system, the third surface comprises skin of a patient, and the analyte includes a liquid-dissolved compound in tissue. In one embodiment of the sensor system, the liquid-dissolved compound is selected from the group consisting of drugs and metabolites. In one embodiment of the sensor system, the third surface comprises a battery. In one embodiment of the sensor system, the third surface comprises a material selected from the group consisting polymeric materials, carbon-fiber materials, composite materials, and multilayered materials.

In yet another aspect, the present invention provides a method for determining a level of a parameter of an analyte. The method can comprise: (a) positioning a probe proximate to a surface to monitor an analyte; (b) positioning a photon source to deliver photons to the probe to excite the probe; (c) positioning a photodetector to receive light emitted by the probe in response to being emitted by the photon source; (d) causing the photon source to direct photons at the probe and excite the probe to emit light in response to receiving the photons; (e) collecting optical data from the photodetector based on the interaction between the light emitted from the probe and the photodetector; (f) determining a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and (g) determining a second level of the parameter of the analyte adjacent the second surface of the probe based on the difference between the first level of the parameter of the analyte and the intermediate level of the parameter of the analyte. In one embodiment of the method, step (g) comprises determining the second level of the analyte adjacent the second surface of the probe by solving a diffusion mathematical problem including the first level of the analyte and the intermediate level of the analyte. In one embodiment of the method, step (g) comprises determining the second level of the analyte adjacent the second surface of the probe by solving an inverse boundary condition mathematical problem including the first level of the analyte and the intermediate level of the analyte. The inverse boundary condition mathematical problem can include a thickness dependent diffusivity variable. The inverse boundary condition mathematical problem can include Dirichlet boundary conditions.

In yet another aspect, the present invention provides a method for determining a level of a parameter of an analyte. The method can comprise: (a) positioning a probe proximate to a surface to monitor an analyte; (b) positioning a photon source to deliver photons to the probe to excite the probe; (c) positioning a photodetector to receive light emitted by the probe in response to being emitted by the photon source; (d) causing the photon source to direct photons at the probe and excite the probe to emit light in response to receiving the photons; (e) collecting optical data from the photodetector based on the interaction between the light emitted from the probe and the photodetector; (f) determining, based on the optical data from the photodetector, a gradient of a parameter of the analyte extending from a first location adjacent the first surface of the probe to an intermediate location between the first location and a second location adjacent the second surface of the probe; and (g) calculating, based on the gradient, a level of the parameter of the analyte adjacent the second surface of the probe.

In one embodiment of the method, the parameter is partial pressure. In one embodiment of the method, the analyte includes at least one of oxygen, carbon dioxide, nitric oxide, and volatile organic compounds. In one embodiment of the method, the analyte includes oxygen.

In one embodiment of the method, the method further comprises: positioning a section of the sensor system over a portion of a third surface to define a zone adjacent the third surface such that the probe is in fluid communication with the zone. In one embodiment of the method, the third surface comprises skin of a patient, and the analyte includes transcutaneous oxygen.

In one embodiment of the method, the probe comprises a layer of a polymeric material embedded with a metalloporphyrin.

In one embodiment of the method, the method further comprises: positioning an oxygen permeable scattering layer between the layer of the polymeric material and the third surface.

In one embodiment of the method, the method further comprises: positioning a semi-permeable layer between the layer of the polymeric material and the photodetector, wherein the semi-permeable layer is semi-permeable to oxygen diffusion therethrough.

In one embodiment of the method, the third surface comprises skin of a patient, the analyte includes transcutaneous oxygen, and an equilibration time of oxygen in the zone is less than 30 minutes when the section of the sensor system is positioned over the portion of the skin, or the equilibration time of oxygen in the zone is less than 20 minutes, or the equilibration time of oxygen in the zone is less than 10 minutes.

In still another aspect, the present invention provides a computer system comprising instructions stored on a non-transitory computer readable medium to cause at least one processor on a computer to: (i) cause a photon source to direct photons at a probe sensitive to an analyte and excite the probe to emit light in response to receiving the photons, the probe having a body including a first surface and an opposite second surface; (ii) receive optical data from a photodetector based on the interaction between the light emitted from the probe and the photodetector; (iii) determine a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and (iv) determine a second level of the parameter of the analyte adjacent the second surface of the probe based on the difference between the first level of the parameter of the analyte and the intermediate level of the parameter of the analyte. In one embodiment of the computer system, the at least one processor determines the second level of the analyte adjacent the second surface of the probe by solving a diffusion mathematical problem including the first level of the analyte and the intermediate level of the analyte. In one embodiment of the computer system, the at least one processor determines the second level of the analyte adjacent the second surface of the probe by solving an inverse boundary condition mathematical problem including the first level of the analyte and the intermediate level of the analyte. The inverse boundary condition mathematical problem can include a thickness dependent diffusivity variable. The inverse boundary condition mathematical problem can include Dirichlet boundary conditions.

In yet another aspect, the present invention provides a computer system comprising instructions stored on a non-transitory computer readable medium to cause at least one processor on a computer to: (i) cause a photon source to direct photons at a probe sensitive to an analyte and excite the probe to emit light in response to receiving the photons, the probe having a body including a first surface and an opposite second surface; (ii) receive optical data from a photodetector based on the interaction between the light emitted from the probe and the photodetector; (iii) determine, based on the optical data from the photodetector, a gradient of a parameter of the analyte extending from a first location adjacent the first surface of the probe to an intermediate location between the first location and a second location adjacent the second surface of the probe, and (iv) calculate, based on the gradient, a level of the parameter of the analyte adjacent the second surface of the probe. In one embodiment of the computer system, the parameter is partial pressure. In one embodiment of the computer system, the analyte includes at least one of oxygen, carbon dioxide, nitric oxide, and volatile organic compounds. In one embodiment of the computer system, the analyte includes oxygen.

### Example

The following Example is provided to demonstrate and further illustrate certain embodiments and aspects of the present invention and is not to be construed as limiting the scope of the invention. The statements provided in the Example are presented without being bound by theory.

### Overview of Example

An important limitation of transcutaneous oxygen monitoring (TCOM) measurements is the need for long equilibration periods in order to obtain stable readings which are close to the true partial oxygen pressure (pO₂) of tissue on the skin surface (transcutaneous pO₂ or tcpO₂). Additionally, the relationship between the obtained readings and the true tcpO₂ is usually not discussed. Upon application of the TCOM devices, an exponential drop is typically observed from atmospheric pO₂ (160 mmHg) to a value in the vicinity of 60-80 mmHg. The speed and amplitude of the decay depends on the volume of trapped air between the detectors and skin, the oxygen diffusion through the different materials used, etc. So far, it has been assumed that given enough time, the obtained pO₂ readings correspond to the real tcpO₂ value. However, if devices make use of semi-breathable materials in order to allow the skin to breathe, the equilibrium pO₂ value which is achieved is an intermediate value between atmosphere and skin pO₂.

Some applications may require avoiding direct contact between skin and O₂-sensing materials, with the addition of semi-breathable layers, further altering the differences between the measured pO₂ and the true tcpO₂. It is, therefore, valuable to greatly reduce or altogether eliminate the waiting times in TCOM devices, and for devices to systematically produce true tissue oxygenation readings, even under changing environmental conditions (temperature, humidity, motion, etc.).

In this Example, we present a toolset to fully leverage a transcutaneous oxygenation monitor (TCOM) wearable technology to accurately measure skin oxygenation values. We describe numerical models and experimental characterization techniques that allow for the extraction of precise tissue oxygenation measurements. The numerical model is based on an inverse boundary problem of the parabolic equation with Dirichlet boundary conditions. To validate this model and characterize the diffusion of oxygen through the oxygen sensing materials, we designed a series of control/calibration experiments modeled after the device's clinical application using oxygenation values in the physiological range expected for healthy tissue. Our results demonstrate that it is possible to obtain accurate tissue pO₂ measurements without the need for long equilibration times with a small wearable device.

### Introduction

As noted above, it would be valuable for TCOM devices to systematically produce true tissue oxygenation readings, while reducing the need for long equilibration times, despite the use of barrier or intermediate layers between tissue and sensors. In this Example, we report the development of a mathematical toolset to address this limitation such that device measured oxygen concentrations can be translated into true oxygenation values while employing materials which can be used for long term wear applications. We describe and test the numerical models along with experimental characterization to create a practical approach to rapidly and simply calculate pO₂ values in future applications.

### Experimental and Numerical Methods

### Transcutaneous Oxygen Monitor Wearable

A recently developed wearable TCOM device [Ref. 12] detects molecular oxygen via changes in the phosphorescence lifetime of an ultrabright metalloporphyrin synthesized in-lab [Ref. 13], which exhibits oxygen quenching of phosphorescence. The change in the phosphor's lifetime T depends on oxygen following the Stern-Volmer relation [Ref. 14]:

$$\frac{{τ}_{0}}{τ} = 1 + {K}_{SV} \times {p O}_{2} \qquad ( 1 )$$

where τ₀ and τ, respectively, are the phosphorescence lifetimes of a probe in the absence and presence of oxygen, K_{SV} is the Stern-Volmer quenching constant which is a function of the lifetime of the probe and its environment (polymeric matrix, solvent, etc.), and pO₂ is the partial pressure of oxygen. This phosphor can be readily embedded within polymer-based films, resulting in ultrathin, breathable films that exhibit bright emission throughout the physiological pO₂ range and are impervious to changes in relative humidity [Ref. 15, 16]. The device improves on existing technology, as it is a true wearable (does not require external readout electronics or cabled leads), it is light and entirely self-contained, and it measures tissue oxygenation without the use of heating elements. The porphyrin has been successfully implemented in different applications with multiple form factors, such as a paintable bandage that has been tested clinically [Ref. 10], wound-care hydrogel dressings [Ref. 17], devices that can be worn on the skin [Ref. 12, 18], intramuscular needles [Ref. 19], and subdermal microneedles [Ref. 20].

In the TCOM wearable device 100 shown in FIGS. 1A and 1B, the oxygen sensing film 105 is composed of: a medical grade semipermeable transparent membrane SP partially occluding the skin from atmospheric oxygen (Bioclusive, Mckesson), a thin poly(propyl methacrylate) layer with the embedded metalloporphyrins forming a probe PPMA, and a highly breathable white scattering layer W which increases the collected phosphorescence signal reaching the photodetector and serves as optical insulation (FIG. 1B, Panel a). The design of the O₂-sensing film 105 allows for short settling or equilibration times due to a conformal, airtight seal over the skin, which minimizes headspace, or the volume of trapped air between device and skin. Additionally, the use of medical grade, semipermeable layer SP permits some moisture and oxygen to diffuse from the skin, allowing the long term wear of the device 100 and materials. The device 100 includes electronics 110 having a controller in communication with the O₂-sensing film 105. The electronics 110 can include a photon source(s), and a photodetector(s). The photon source(s) can include one or more photon sources (e.g., one, two, three, four), and in some cases, the photon source(s) can include a single photon source. Each photon source can be optically coupled to the probe PPMA and can be configured to emit respective light towards the probe PPMA, which causes the probe PPMA to emit light in response to absorbing the light. For example, the light can interact with a photoluminescent material of the probe PPMA to emit photoluminescent light. As a more specific example, the light can interact with a phosphorescent material of the probe PPMA to emit phosphorescent light. The wearable device described in Ref. 12 is attached to the O₂-sensing film 105 and excites the phosphorescence of the porphyrins in the PPMA layer via two ultraviolet LEDs, with the phosphorescence detected via a small photodiode. A band 120 couples the electronics 110 to a body part such as a leg shown in FIG. 1A, and an arm shown in FIG. 1B.

### Inverse Boundary Problem of the Parabolic Equation with Dirichlet Boundary Conditions

An important detail for the present Example is that the measurement of oxygen partial pressure is obtained from porphyrins embedded within the PPMA layer, which is sandwiched between two other layers and is also not in direct contact with the patient's skin. Looking at FIG. 1B Panel b, the films and devices provide pO₂ readings which are at an intermediate concentration between atmosphere on one side of the O₂-sensing film 105 and the tissue tcpO₂ on the other. This scenario corresponds to a diffusion problem in which the pO₂ is known at one boundary (atmosphere on the left—e.g., LBC=160 mmHg) and one intermediate point (pO₂ in the middle of the film—e.g., TCOM reading), and we wish to know the pO₂ at the other boundary (the right, tissue in this case—e.g., RBC=???). This specific type of mathematical problem is known as an inverse boundary condition problem. In this Example, we develop and demonstrate a one-dimensional inverse boundary problem solution along with control experiments to estimate true tissue oxygenation from the readings obtained with the oxygen sensing film and TCOM wearable technology.

In this case, the goal is to determine the concentration u, or value of pO₂, at one boundary; therefore, one needs to solve the inverse boundary problem of the parabolic equation with a thickness dependent diffusivity, D(x):

$$\frac{\partial \text{?}}{\partial t} = \frac{\partial }{\partial x} ( D ( x ) \frac{\partial \text{?}}{\partial x} ) , 0 < x < l , 0 < t \leq T \qquad ( 2 )$$

$$with Dirichlet boundary conditions :$$

$$u , ( 0 , t ) = μ ( t ) , u ( \text{?} , t ) = ρ ( t ) \qquad ( 3 )$$

$$\text{?} \text{indicates text missing or illegible when filed}$$

where μ(t) can be a constant equal to the atmospheric value of oxygen partial pressure (160 mmHg at sea level) and μ(t) is the value of the function at the right boundary, which is unknown. The use of Dirichlet, or first-type, boundary conditions is important, as the goal is to obtain the value of the solution at the boundary, in this case the skin surface.

The additional condition at the intermediate point x_{*} is

$$u ( x \text{?} , \text{?} ) = φ ( t ) , 0 < t \leq T \qquad ( 4 )$$

$$\text{?} \text{indicates text missing or illegible when filed}$$

which corresponds to the pO₂ measurement obtained using the wearable device, pO₂^{TCOM}

Finally, the initial condition is set as

$$u ( x , 0 ) = g ( x ) , 0 < x < \text{?} \qquad ( 5 )$$

$$\text{?} \text{indicates text missing or illegible when filed}$$

which will typically correspond to an initial pO₂ value of atmospheric oxygenation for all x, i.e., g(x)=160 mmHg. This initial condition corresponds to a sensor material that has not yet been attached to the surface of skin.

### Discrete Inverse Boundary Problem

The method, described in detail in Ref. 21, comprises discretizing the problem stated in equations 2-5 using the finite difference method, and including a diffusivity D that is x dependent. Here, we have modified this algorithm, which used a Neumann left boundary condition, to instead include Dirichlet boundary conditions, as the problem under study in the Example (transcutaneous oxygenation) requires specifying the value of the concentration at the boundary condition, rather than its derivative.

The space and time coordinates x and t have the following nodes:

$$ω \text{?} = \text{?} {x}_{i} = ih , i = 1 , 2 , … , {N}_{x} - 1 , h = \text{?} / {N}_{x \text{?}}$$

$$ω \text{?} = \text{?} = j τ , j = 1 , 2 , … , N \text{?} , τ = T / {N}_{t} \text{?}$$

$$\text{?} \text{indicates text missing or illegible when filed}$$

The node corresponding to the observation x_{*} for the additional condition is defined as x_{*}=n_{*}h. For brevity, we will denote

$${y}_{i} - {y}_{i}^{j} , {\breve{y}}_{i} = {y}_{i}^{j - 1} ,$$

and the same notation will be used for time dependent functions such as μ(t) or φ(t), e.g., μ(t)=μ^{j}=μ. Also, to express the problem using an integro-interpolation method, we define:

$$\text{?}$$

$$\text{?}$$

$$\text{?} \text{indicates text missing or illegible when filed}$$

On the grid ωh_{τ}=ωₕ×ω_{τ}, Ref. 21 employs the implicit difference scheme for the approximation by time and the finite difference method for the boundary inverse problem. Then, for t∈ω_{τ}, the discrete problem is expressed as

$$\frac{{y}_{i} - {{y}^{︶}}_{i}}{h τ} = ( {α}_{i} ( {y}_{i} - {y}_{i - 1} ) - {α}_{i + 1} ( {y}_{i + 1} - {y}_{i} ) ) \qquad ( 6 )$$

with i=1, . . . , Nₜ−1.

The left boundary is then modified from the method in Ref. 21 and left Dirichlet boundary defined as

$$\frac{2 ( {y}_{0} - {{y}^{︶}}_{0} )}{h τ} = ( {α}_{1} ( {y}_{1} - 2 {y}_{0} + μ ) \qquad ( 7 )$$

with the additional condition

$${y}_{{n}_{*}} = φ \qquad ( 8 )$$

and the initial conditions.

$${y}_{i}^{0} = {g}_{i} \qquad ( 9 )$$

### Algorithm for the Inverse Problem Solution

To solve the inverse boundary problem stated above, the following linear algebraic equations must be solved at each time point tⱼ. As is discussed in Ref. 21, the function yᵢ is decomposed in two grid functions Ref. 22:

$${y}_{i} = {v}_{i} + {y}_{n} \cdot {w}_{i} , i = 0 , … , n \qquad ( 10 )$$

with n=Nₓ in this case. The values of vᵢ at each time point tⱼ can be determined by solving the following system of linear equations. The first case or line below is the only change with respect to the work in Ref. 21, as it depends on the left boundary condition.

$$\{ \begin{matrix}2 c ( {v}_{0} - {{y}^{︶}}_{0} ) = {a}_{1} ( {v}_{1} - 2 {v}_{0} - μ ) \\ 2 c ( {v}_{i} - {{y}^{︶}}_{i} ) = {a}_{i} ( {v}_{i + 1} - {v}_{i} ) - {a}_{i} ( {v}_{i} - {v}_{i - 1} ) \\ {v}_{n} = 0\end{matrix} \qquad ( 11 )$$

$$with c = {h}^{2} / {2}_{T} .$$

The system of equations to obtain the auxiliary unknowns wᵢ is unchanged with respect to Ref 21.

$$\{ \begin{matrix}{cw}_{0} = {a}_{1} ( {w}_{0} - {w}_{1} ) \\ {w}_{i} = {a}_{i + 1} ( {w}_{i + 1} - {w}_{i} ) - {a}_{i} ( {w}_{i} - {w}_{i - 1} ) \\ {w}_{n} = 1\end{matrix} \qquad ( 12 )$$

At each time point tⱼ, after the vᵢ and wᵢ values are known, the values of the function at the right boundary yₙ are calculated using y_{n*}=φ and y_{n*}=v_{n*}+y_{n*}w_{n*} so that

$${y}_{n} = ( φ - {v}_{{n}_{*}} / {w}_{{n}_{*}} ) \qquad ( 13 )$$

Finally, the applicability of the algorithm requires wₙ≠0 (or not too small), which is satisfied when the Courant stability criterion for the parabolic equation, defined as C=τ/2h², is sufficiently large, i.e., C>>1 [Ref. 21].

### Testing the Algorithm

As the starting point, we first reproduced the inverse boundary problem algorithm reported in Ref. 21, which used a Neumann left boundary condition. To test the algorithm, we then solved the following sample problem described in the reference. First the direct initial-boundary value problem was implemented employing the implicit difference scheme using GNU Octave [Ref. 23] to solve the sample problem, which comprises:

$$\frac{\partial u}{\partial t} = \frac{{\partial }^{2} u}{\partial {x}^{2}} , 0 < x < l , 0 < t \leq T \qquad ( 14 )$$

$$with the left boundary :$$

$$\frac{\partial u ( 0 , t )}{\partial x} = 0 , u ( {x}_{*} , t ) = φ ( t ) , 0 < t \leq T \qquad ( 15 )$$

$$the following initial condition :$$

$$u ( x , 0 ) = 0 \qquad ( 16 )$$

$$and the right boundary condition having the form :$$

$$ρ ( t ) = \{ \begin{matrix}2 t / T & 0 \leq t < T / 2 \\ 2 ( 1 - t / T ) & T / 2 \leq t \leq T\end{matrix} \qquad ( 17 )$$

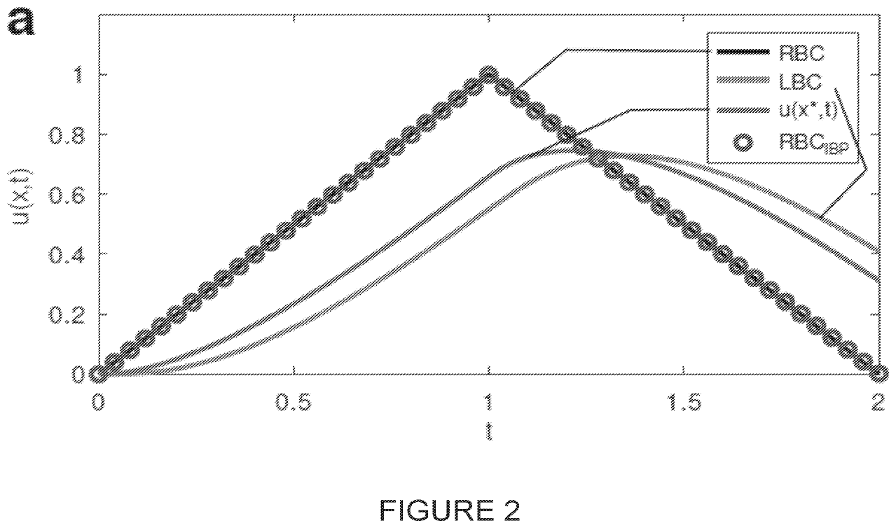

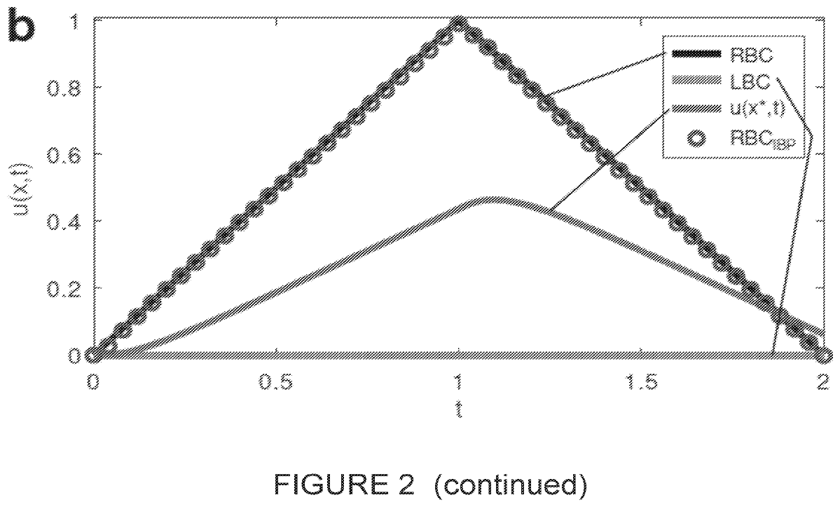

For the calculations, the following parameters were used: I=1, Nₓ=100, T=2, Nₜ=100. We then define the additional condition for the inverse boundary problem u(x_{*}, t)=φ(t) with x_{*}=0.5. The left and right boundaries as well as φ(t) are plotted as solid lines in FIG. 2 Panel a.

We can now use the calculations from the direct model, namely the left boundary and φ(t) as the additional condition, to compute the right boundary using the inverse boundary problem algorithm from Ref. 21.

The calculated right boundary is plotted as scatter points in FIG. 2 Panel a, which matches the known function ρ(t) from the direct model, as well as the result from the reference article.

With this complete, it is now possible to test the modified algorithm described above to solve the inverse boundary problem for the parabolic equation with left Dirichlet boundary conditions. This was solved as above, except the left boundary was now the following:

$$u ( 0 , t ) = 0 \qquad ( 18 )$$

The same parameters were once again used: I=1, Nₓ=100, T=2, Nₜ=100, C=100 and u(x_{*}, t)=φ(t) with x_{*}=0.5. We solve the direct problem, with the known left and right boundaries and yields φ(t), plotted (as solid lines) in FIG. 2 Panel b.

Finally, the modified inverse boundary problem is able to reconstruct the right boundary, plotted as scatter points in FIG. 2 Panel b, to match closely the known function ρ(t) from the direct model, as in the case with Neumann boundary conditions.

### Inverse Boundary Problem Applied to Transcutaneous Oxygenation

The previous examples deal with a diffusion coefficient which is constant for all xᵢ. In order to fully describe the multilayer system of the oxygen sensing film, a three dimensional model should be used along with a diffusivity that changes with depth, i.e., D(z). However, as explained below, the use of a conservative finite difference scheme of the 1D diffusion equation [Ref. 24] is sufficient for our case.

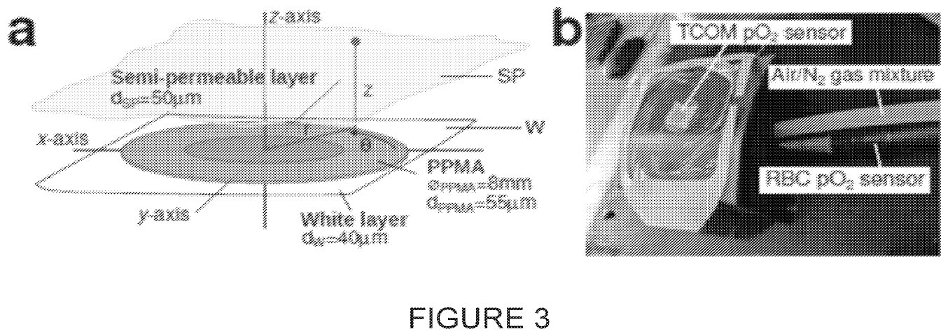

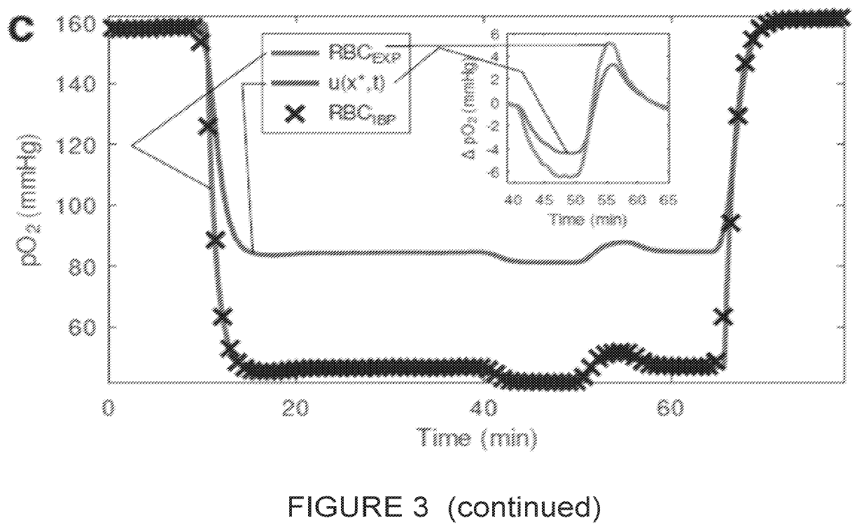

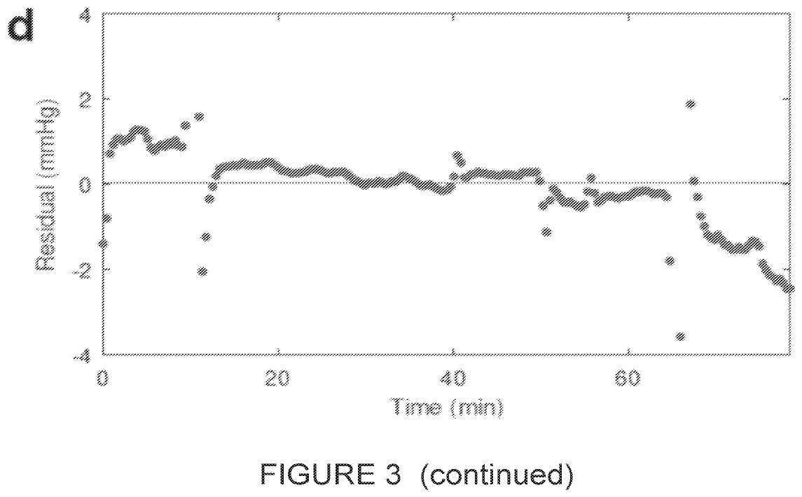

As seen in FIG. 3 Panel a, the diameter of the PPMA oxygen-sensing disk is ø_{PPMA}=8 mm, while its thickness (measured with a caliper) was found to be d_{PPMA}=55 μm. The highlighted central region of the PPMA disk, with an approximate diameter of ø=4 mm is the part of the film that is excited by the LEDs in the wearable device [Ref. 12], and therefore provides the pO₂ readings. The semipermeable film SP is much larger than the PPMA disk (see a photo of a film in FIG. 3 Panel b), and it was found to have a thickness of d_{SP}=50 μm. The white scattering layer W has a thickness of d_{W}=45 μm and covers the entire PPMA disk.

Given that only the central part of the PPMA disk is probed to measure pO₂ and that the thickness of the films is much smaller than their width (ø_{PPMA}>>d_{PPMA}), the model can be simplified by assuming oxygen diffusion only in direction normal to the film plane (through the different layers) and neglect any in-plane or side diffusion. In this case, if we consider the parabolic equation in cylindrical (ρ, φ, z) rather than Cartesian coordinates (x, y, z), and diffusion only depends on depth z, and we have that the oxygen concentration within the film structure is u(z, t) and the diffusivity D(z), the three-dimensional problem is reduced to a problem in one dimension with only perpendicular diffusion of oxygen through the different layers of the film.

The diffusion equation in three dimensions is

$$\frac{\partial u}{\partial t} = ∇ \cdot ( D \cdot ∇ u ) = ∇ D ∇ u + D \cdot {∇}^{2} u \qquad ( 19 )$$

$$In cylindrical coordinates , we have$$

$$∇ f = \frac{\partial f}{\partial r} \hat{r} + \frac{1}{r} \frac{\partial f}{\partial ϕ} \hat{ϕ} + \frac{\partial f}{\partial 𝓏} \hat{𝓏} \qquad ( 20 )$$

$${∇}^{2} f = \frac{1}{r} \frac{\partial }{\partial r} ( r \frac{\partial f}{\partial r} ) + \frac{1}{{r}^{2}} \frac{{\partial }^{2} f}{\partial {ϕ}^{2}} + \frac{{\partial }^{2} f}{\partial {𝓏}^{2}} \qquad ( 21 )$$

$$then given u = f ( 𝓏 , t ) and D = g ( 𝓏 ) , eq 19 becomes$$

$$\frac{\partial u}{\partial t} = \frac{\partial D}{\partial 𝓏} \frac{\partial u}{\partial 𝓏} + D \frac{{\partial }^{2} u}{\partial {𝓏}^{2}} \qquad ( 22 )$$

which is equivalent to eq 2, the one-dimensional diffusion equation with a z dependent diffusivity. For convenience, we will relabel the depth z as x. We also have left Dirichlet boundary conditions

$$u ( 0 , t ) = {p O}_{2}^{atm} = 160 mmHg \qquad ( 23 )$$

and the additional condition at the point x=x_{*} corresponding to the pO₂ measured by our device, with x_{*} being somewhere inside the PPMA oxygen sensing film

$$u ( {x}_{*} , t ) = {{p O}_{2} ( t )}^{TCOM} \qquad ( 24 )$$

It should be noted that considering the emission of molecules to arise at a specific depth of the PPMA layer is a simplification. In the wearable sensors, this measurement reflects the average value of the oxygenation in the entire volume of the PPMA layer excited by the LEDs and sampled by the photodiode. Lastly, we propose a smooth (differentiable) D(x) function to have a thickness dependent diffusion coefficient, to account for the different layers making up the O₂-sensing film, using the measured layer thicknesses. To create smooth (differentiable) but sharp transitions in diffusivity to reflect changes between the layers, we use the following function:

$$D ( x ) = arctan \vert A \cdot \frac{( x - {d}_{1} )}{max ( x )} + π / 2 \vert \cdot \frac{( {D}_{2} - {D}_{t} )}{π} + {D}_{1} \qquad ( 25 )$$

where D₁ and D₂ are the diffusivity values of layers 1 and 2, d₁ is the thickness of layer 1. Since the film has three layers, a piece-wise function is created using the previous equation twice. The constant A can be used to tweak the sharpness of the step, in our case, A=5000.

### Results And Discussion

### Control Experiment and Modeling

In order to estimate the unknown diffusion coefficients of the three layers SP, PPMA, W, and to solve the inverse boundary problem, we designed an experiment in which pO₂ in the left boundary (atmosphere), intermediate value (TCOM reading) and the right boundary (oxygenation under the film) are known. For this experiment, shown in FIG. 3 Panel b, we designed and 3D printed a calibration block made of a rubber-like photopolymer using a Formlabs Form 3B printer. The block had an inner chamber which could be connected with tubing and filled with a controlled gas mixture. An additional opening allowed sampling the pO₂ of the gas mixture with a commercial oxygen sensor (Presens). An area of diameter 1 cm with small perforations was designed on the surface of the block, over which the oxygen-sensing film is placed, so the bottom of the film is exposed to the pO₂ of the inner chamber.

As shown in FIG. 3 Panel b, the films are adhered onto the calibration block, mimicking their clinical use, and the wearable device is then attached to the film to obtain pO₂ readings of the PPMA layer, or pO₂TCOM. The commercial sensor probes the pO₂ of the gas mixture, or RBC_{EXP}.

The pO₂ of the gas mixture was modified by varying the mix of nitrogen and air being fed into an automated gas mixer [Ref. 25], which was programmed to simulate clinical results from the following protocol: (a) the device probes atmospheric pO₂; (b) the device is put on a subject's skin, and allowed to equilibrate for 20-30 minutes; (c) blood flow is restricted during ˜10 minutes via a pressure cuff, showing a decrease in oxygenation; (d) upon release of the cuff and restoration of blood flow, an increase in pO₂ is observed (reactive hypereamia), after which pO₂ settles at the initial value before cuffing; and (e) device is removed from the subject's arm and re-exposed to atmosphere.

Both pO₂^{TCOM} and RBC_{EXP} are shown in FIG. 3 Panel c. Five different films were tested in this system, the first of which is shown in FIG. 3 Panel c. The inset shows how the change in pO₂ is underestimated by the raw TCOM reading compared to the right boundary measurement during the period emulating the restriction or occlusion of blood flow.

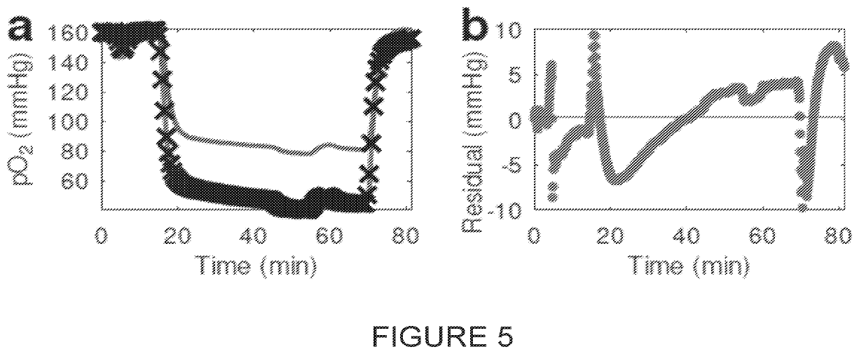

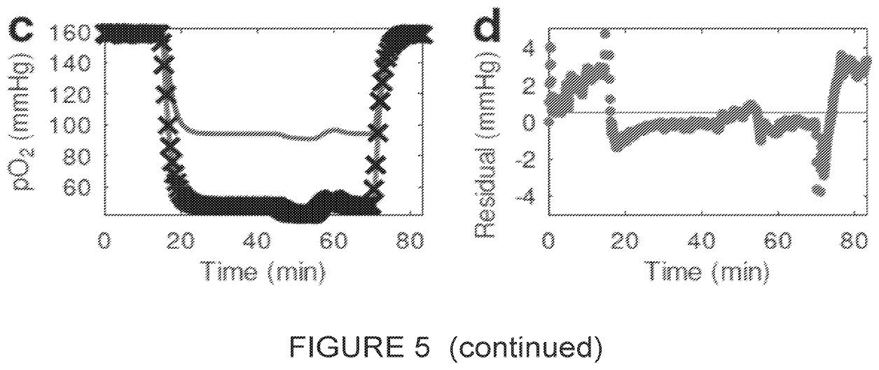

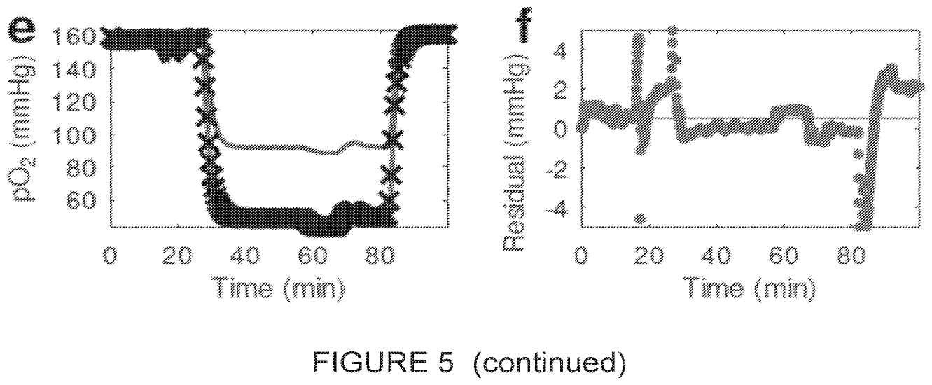

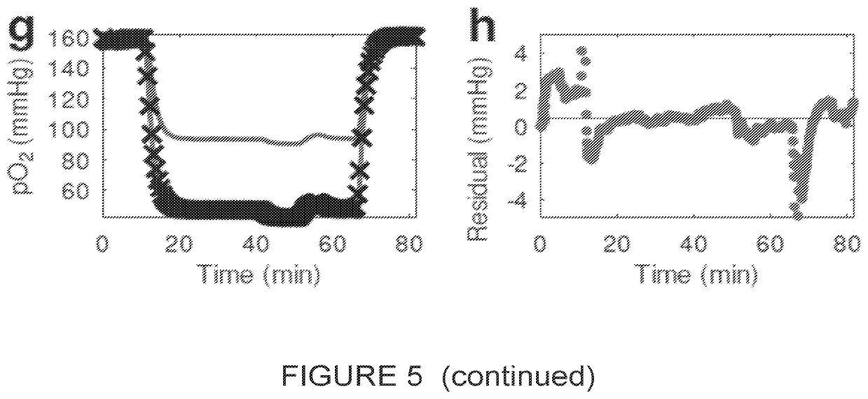

We then fit the data with the inverse diffusion equation model with a thickness dependent diffusion coefficient defined by the piece-wise function described above. For all inverse boundary calculations mentioned, we used the parameters: I=145 μm, Nₓ=1000, T=78.5 min, Nₜ=200, which yields a Courant stability criterion of C=9.341. The result of the algorithm was of equal quality as for C=93.41 and considerably faster to calculate. Using the known left boundary and intermediate reading pO₂^{TCOM}, we use a nonlinear least squares algorithm to match the reconstructed right boundary to the experimentally measured value (RBC_{EXP}). This model uses four fitting coefficients: the value of x_{*,} as well as the diffusivity values of the three layers D_{SP}, D_{PPMA}, and D_{W}. The result of the fit is shown in FIG. 3 Panel c, with the reconstructed right boundary matching well the experimental values. A residual plot in FIG. 3 Panel d shows further evidence of the goodness of the fit. The results of the fit and residual for the remaining three films are shown in FIG. 5.

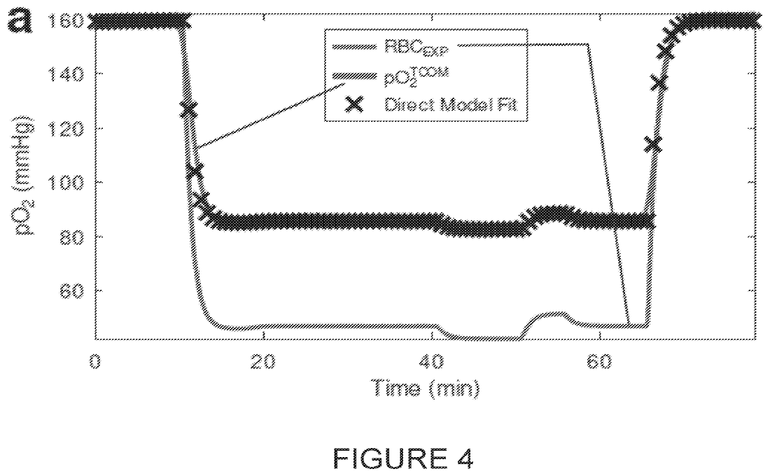

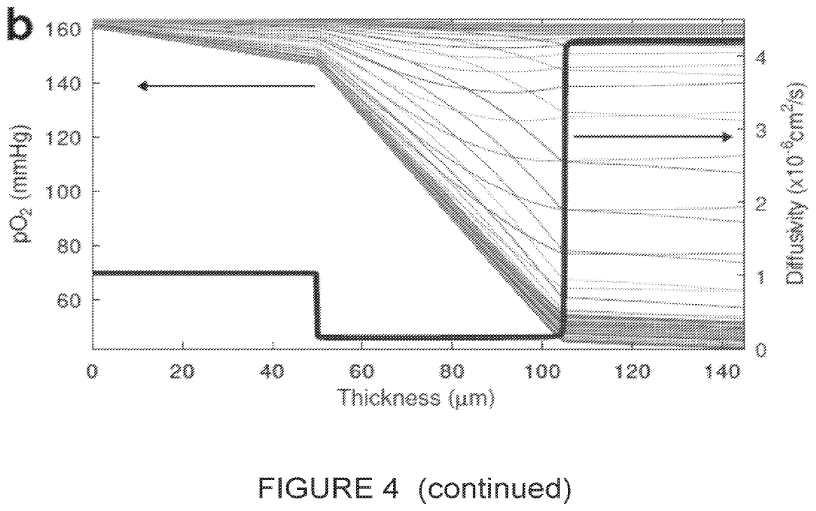

The values of the fitting coefficients obtained for all films are shown in Table 1, with Film #1 corresponding to the results shown in FIGS. 3 and 4.

TABLE 1
Fitting Coefficients Obtained from the Control
Experiment from Five Different Films

| Film# | x* (μm) | D_{SP} (cm²/s) | D_{PPMA} (cm²/s) | D_{W} (cm²/s) |
|---|---|---|---|---|
| 1 | 80.6 | 1.50 × 10⁻⁶ | 1.40 × 10⁻⁷ | 1.62 × 10⁻⁵ |
| 2 | 79.8 | 2.71 × 10⁻⁷ | 1.14 × 10⁻⁷ | 7.28 × 10⁻⁶ |
| 3 | 80.9 | 8.33 × 10⁻⁶ | 8.22 × 10⁻⁸ | 9.93 × 10⁻⁶ |
| 4 | 81.4 | 8.33 × 10⁻⁶ | 9.12 × 10⁻⁸ | 1.66 × 10⁻⁵ |
| 5 | 80.5 | 8.33 × 10⁻⁰ | 1.01 × 10⁻⁷ | 1.63 × 10⁻⁵ |
| X̅ | 80.7 | | 1.05 × 10⁻⁷ | 1.33 × 10⁻⁵ |
| σ_{X} | 0.6 | | 2.25 × 10⁻⁸ | 4.37 × 10⁻⁶ |

Of these, the most consistent value is that of x_{*,} which shows a mean value of 80.7±0.6 μm, close to the middle of the PPMA film (77.5 μm). For the semipermeable layer SP, the coefficient D_{SP} reached the upper bound set for the fitting algorithm on three of the films so the mean and standard deviation is not calculated. The values for films 1 and 2 fall within the experimentally observed range at room temperature 10⁻⁸-10⁻⁶ cm²/s [Ref. 26,27]. For PPMA, the mean value is on the high end of the known range, (2-8)×10⁻⁸ cm²/s [Ref. 28-30], although it has a large relative error, (1.05±0.22)×10⁻⁷ cm²/s. The breathable, white scattering layer W, composed of dimethylsiloxane and TiO₂ powder [Ref. 12, 19], yields a mean diffusivity of (1.33±0.44)×10⁻⁶ cm²/s which matches experimental values [Ref. 31] but also has a large relative error, (1.33±0.44)×10⁻⁶ cm²/s. We also fit an exponentially decaying function (f(t)=A₁+A₂·e^{-t/T}) to the initial drop from atmospheric pO₂ down to the equilibrium value, and obtained an average decay time for all five films of τ=2.25±0.08 minutes for the TCOM data and T=1.7±0.1 minutes for the RBC_{EXP} readings. This points to our films having fast settling times.

For completeness, as in the examples discussed in FIG. 2, we have already solved the inverse problem so we will check if solving the direct problem will produce values which match the experiment. We fed the fitting coefficients to a direct model with thickness dependent diffusivity, and we can see that with the known left and right boundaries, the calculated oxygen concentration at x_{*} matches the experimental results, pO₂^{TCOM}, as seen in FIG. 4 Panel a. The diffusivity profile or D(x) is also shown in FIG. 4 Panel b, along with the O₂ concentration gradient through the layers.

### Applying the Algorithm to Clinical Data

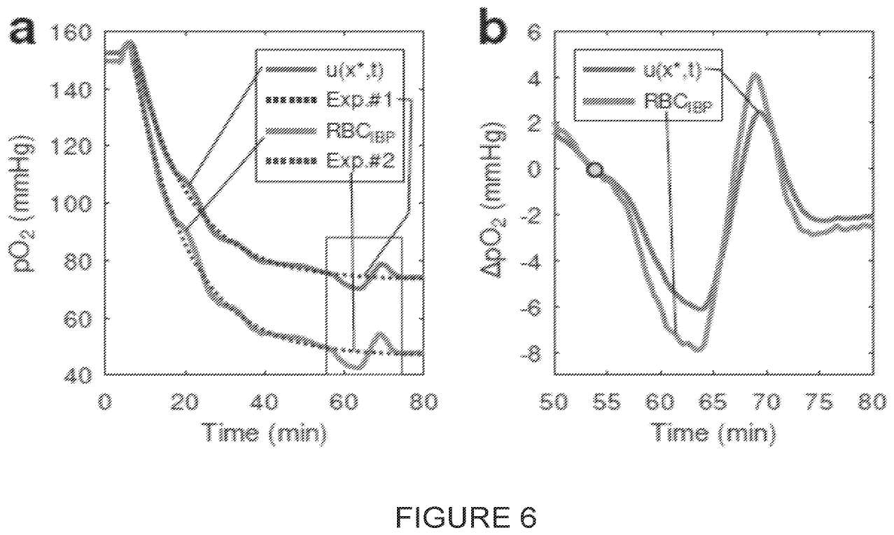

The inverse boundary problem algorithm, now tuned to the specific diffusion properties of the O₂-sensing films, can be used to calculate the “true” skin pO₂ using the TCOM oxygenation readings. FIG. 6 shows the result of applying the algorithm to a clinical study measurement. The measurement was carried out following the protocol: (a) the device probes atmospheric pO₂; (b) the device is put on a subject's lower leg (calf), and allowed to equilibrate for ˜40 minutes; (c) blood flow is restricted for 10 minutes via a pressure cuff on the thigh, resulting in decreased oxygenation; (d) release of the cuff results in a reactive hypereamia with concomitant an increase in pO₂ and subsequent return of pO₂ to initial values.

As can be seen in the FIG. 6 Panel a, the IBP pO₂ readings equilibrate from atmospheric pO₂ down to tissue levels, within 30 minutes of application. The fitting of an exponential function to both time series yields an exponential decay time of ˜13 minutes for both TCOM and IBP readings (13.56 and 13.43 minutes respectively). The difference with respect to the value obtained above for films on the calibration block could arise from trapped pockets of air in wrinkles of the adhesive film when sealing over the skin, or a response of the tissue itself.

The equilibrium pO₂ value achieved of around 50 mmHg is in the range of what is expected of healthy tissue [Ref. 6]. Further, FIG. 6 Panel b plots the relative change in oxygenation for the TCOM and IBP readings during the application of the pressure cuff (the pO₂ value when the pressure cuff is applied is subtracted from the measurement). The graph shows how the pO₂ changes due to increasing/decreasing blood flow are sharper and of larger amplitude than in the raw readings reported by the TCOM device, and estimates more accurately the real drop in oxygenation of the tissue.

### CONCLUSIONS

The results show that we are able to numerically obtain pO₂ values which are closer in value to those of tissue and quantitatively reflect the changes in oxygenation on the skin surface, by characterizing the oxygen gradient through the O₂-sensing film's layers, allowing us to measure precise changes in oxygenation when blood flow is altered.

In this Example, we designed the methodology (numerical and experimental) to characterize the diffusion of oxygen through multilayer films and materials. It is interesting to note that we also observed, in the same experimental setup, results from different films that were qualitatively similar but showed minor quantitative differences. For example, individual films were found to display different equilibrium pO₂ values measured by the device. This translated to some of the fitting parameters in Table 1 not yielding values consistent between films (such as D_{SP}). These differences are likely due to human factors, as the films are fabricated by hand. For example, films could present differences in trapped pockets of air between the layers. It is also possible that there may be different exposure of the left boundary to atmospheric air due to an adhesive ring support used to attach the measurement device to the film. Differences in reported pO₂ between films could be caused by experimental factors such as an imperfect seal of the film on the chamber. The approach developed here can be used to study film construction effects and improve not only the sensor film materials, but also the consistence in their construction. For example, left boundary exposure differences could be addressed by adding holes on the sensor head to ensure a direct contact of atmospheric air with the top of the film.

The apparent higher diffusivity of PPMA measured in this Example as compared to the literature may be due to changes in pore size from embedding the metalloporphyrin [Ref. 30], or may arise from the appearance of cracks in the glassy PPMA from manipulation while stacking the layers. However, determining diffusion coefficients was not undertaken, as we have proposed a model of the system under study. These values can and should be measured experimentally.

We found parameter values of dx and dt, which ensure a stable calculation (Courant stability criterion C>1) while at the same time resulting in a fast but accurate calculation. The algorithm could be embedded in the device's firmware to calculate tissue pO₂ real-time after each pO₂ sample, as the sampling times used can range from the order of seconds to minutes. A real-time implementation of this algorithm can also drastically reduce settling or equilibration times for the devices to report on skin pO₂ values.

We contemplate the improvement of the algorithm to more accurately predict tissue oxygenation. For example, the algorithm could consider the pO₂^{TCOM} signal as arising from the phosphorescence of the entire PPMA layer, as opposed to a specific depth (x_{*} in the model), or consider the changes in the diffusivity of the materials with temperature. The model could also be expanded by adding a simulation of skin oxygen consumption and transport [Ref. 32] to remove the effect of the low permeability of epidermis and obtain a value closer to that of subdermal tissue.

### REFERENCES

1. Vegfors, M.; Lindberg, L. G.; Lennmarken, C., “The influence of changes in blood flow on the accuracy of pulse oximetry in humans”, Acta Anaesthesiol. Scand. 1992, 36, 346-349.
2. Li, Z.; Navarro-Alvarez, N.; Keeley, E. J.; Nowell, N. H.; Goncalves, B. M.; Huang, C. A.; Evans, C. L., “Non-invasive monitoring of skin inflammation using an oxygen-sensing paint-on bandage”, Biomedical optics express 2017, 8, 4640-4651.
3. McMillan, T. E.; Gardner, W. T.; Schmidt, A. H.; Johnstone, A. J., “Diagnosing acute compartment syndrome—where have we got to?”, Int. Orthop. 2019, 43, 2429-2435.
4. McPhail, L. R.; Cooper, L. T.; Hodge, D. O.; Cabanela, M. E.; Rooke, T. W., “Transcutaneous partial pressure of oxygen after surgical wounds”, Vasc. Med. 2004, 9, 125-127.
5. Moosa, H. H.; Makaroun, M. S.; Peitzman, A. B.; Steed, D. L.; Webster, M. W., “TcPO₂ values in limb ischemia: Effects of blood flow and arterial oxygen tension”, Journal of Surgical Research 1986, 40, 482-487.
6. Baumgartl, H.; Ehrly, A.; Saeger-Lorenz, K.; Lübbers, D., “Clinical Oxygen Pressure Measurement: Tissue Oxygen Pressure and Transcutaneous Oxygen Pressure; Springer, 1987; pp 121-128.
7. Holst, G. A.; Koster, T.; Voges, E.; Lübbers, D. W., “FLOX—an oxygen-flux-measuring system using a phase-modulation method to evaluate the oxygen-dependent fluorescence lifetime”, Sensors and Actuators: B. Chemical 1995, 29, 231-239.
8. Stücker, M.; Struk, A.; Altmeyer, P.; Herde, M.; Baumgartl, H.; Lübbers, D. W., “The cutaneous uptake of atmospheric oxygen contributes significantly to the oxygen supply of human dermis and epidermis”, Journal of Physiology 2002, 538, 985-994.
9. Babilas, P.; Lamby, P.; Prantl, L.; Schreml, S.; Jung, E. M.; Liebsch, G.; Wolfbeis, O. S.; Landthaler, M.; Szeimies, R.-M.; Abels, C., “Transcutaneous pO₂ imaging during tourniquet-induced forearm ischemia using planar optical oxygen sensors”, Skin Research and Technology 2008, 14, 304-311.
10. Marks, H.; Bucknor, A.; Roussakis, E.; Nowell, N.; Kamali, P.; Cascales, J. P.; Kazei, D.; Lin, S. J.; Evans, C. L., “A paintable phosphorescent bandage for postoperative tissue oxygen assessment in DIEP flap reconstruction”, Science Advances 2020, 6, No. eabd1061.
11. Alford, R.; Simpson, H. M.; Duberman, J.; Hill, G. C.; Ogawa, M.; Regino, C.; Kobayashi, H.; Choyke, P. L., “Toxicity of organic fluorophores used in molecular imaging: literature review”, Molecular imaging 2009, 8, 6.
12. Cascales, J. P.; Roussakis, E.; Witthauer, L.; Goss, A.; Li, X.; Chen, Y.; Marks, H. L.; Evans, C. L., “Wearable device for remote monitoring of transcutaneous tissue oxygenation”, Biomed. Opt. Express 2020, 11, 6989-7002.
13. Roussakis, E.; Li, Z.; Nowell, N. H.; Nichols, A. J.; Evans, C. L., “Bright, “clickable” porphyrins for the visualization of oxygenation under ambient light “, Angew. Chem., Int. Ed. 2015, 54, 14728-14731.
14. Stern, O.; Volmer, M. Über die abklingzeit der fluoreszenz”, Phys. Z. 1919, 20, 183-188.
15. Roussakis, E.; Cascales, J. P.; Marks, H. L.; Li, X.; Grinstaff, M.; Evans, C. L., “Humidity-Insensitive Tissue Oxygen Tension Sensing for Wearable Devices”, Photochem. Photobiol. 2020, 96, 373-379.
16. Li, X.; Roussakis, E.; Cascales, J. P.; Marks, H. L.; Witthauer, L.; Evers, M.; Manstein, D.; Evans, C. L., “Optimization of bright, highly flexible, and humidity insensitive porphyrin-based oxygen-sensing materials”, Journal of Materials Chemistry C 2021, 9, 7555-7567.
17. Marks, H. L.; Cook, K.; Roussakis, E.; Cascales, J. P.; Korunes-Miller, J. T.; Grinstaff, M. W.; Evans, C. L., “Quantitative Luminescence Photography of a Swellable Hydrogel Dressing with a Traffic-Light Response to Oxygen (Adv. Healthcare Mater. 10/2022)”, Adv. Healthcare Mater. 2022, 11, 2270048.
18. Cascales, J. P.; Greenfield, D. A.; Roussakis, E.; Witthauer, L.; Li, X.; Goss, A.; Evans, C. L., “Wireless wearable sensor paired with machine learning for the quantification of tissue oxygenation”, IEEE Internet of Things Journal 2021, 8, 17557-17567.
19. Witthauer, L.; Cascales, J. P.; Roussakis, E.; Li, X.; Goss, A.; Chen, Y.; Evans, C. L., “Portable oxygen-sensing device for the improved assessment of compartment syndrome and other hypoxiarelated conditions”, ACS sensors 2021, 6, 43-53.
20. Müller, M.; Cascales, J. P.; Marks, H. L.; Wang-Evers, M.; Manstein, D.; Evans, C. L., “Phosphorescent Microneedle Array for the Measurement of Oxygen Partial Pressure in Tissue”, ACS Sensors 2022, 7, 3440-3449.
21. Vasilev, V.; Vasilyeva, M.; Kardashevsky, A., “The numerical solution of the boundary inverse problem for a parabolic equation”, AIP Conf. Proc. 2016, 100010.
22. Vabishchevich, P.; Vasilev, V., “Computational determination of the lowest order coefficient in a parabolic equation”, Doklady Mathematics 2014, 89, 179-181.
23. Eaton, J. W.; Bateman, D.; Hauberg, S.; Wehbring, R. GNU Octave version 3.8.1 manual: a high-level interactive language for numerical computations; CreateSpace Independent Publishing Platform, 2014; ISBN 1441413006.
24. Lee, J.; Jeong, M. S., Stability of Finite Difference Schemes on the Diffusion Equation with Discontinuous Coefficients; Massachusetts Institute of Technology, 2017.
25. Cascales, J. P.; Li, X.; Roussakis, E.; Evans, C. L., “A Patient-Ready Wearable Transcutaneous CO2 Sensor”, Biosensors 2022, 12, 333.
26. Wang, Y.; Gupta, M.; Schiraldi, D. A., “Oxygen permeability in thermoplastic polyurethanes”, J. Polym. Sci., Part B: Polym. Phys. 2012, 50, 681-693.
27. Coelho, I.; Pires, R. F.; Gonçalves, S. B.; Bonifacio, V. D.; Faria, M., “Gas Permeability and Mechanical Properties of Polyurethane-Based Membranes for Blood Oxygenators”, Membranes 2022, 12, 826.
28. Klinger, M.; Tolbod, L. P.; Gothelf, K. V.; Ogilby, P. R., “Effect of polymer cross-links on oxygen diffusion in glassy PMMA films”, ACS Appl. Mater. Interfaces 2009, 1, 661-667.
29. Poulsen, L.; Ogilby, P. R., “Oxygen diffusion in glassy polymer films: effects of other gases and changes in pressure”, J. Phys. Chem. A 2000, 104, 2573-2580.
30. Hu, C.-C.; Liu, T.-C.; Lee, K.-R.; Ruaan, R.-C.; Lai, J.-Y., “Zeolite-filled PMMA composite membranes: influence of coupling agent addition on gas separation properties”, Desalination 2006, 193,
   14-24.
31. Merkel, T.; Bondar, V.; Nagai, K.; Freeman, B.; Pinnau, I, “Gas sorption, diffusion, and permeation in poly (dimethylsiloxane)”, J. Polym. Sci., Part B: Polym. Phys. 2000, 38, 415-434.
32. Wang, W., “Oxygen partial pressure in outer layers of skin: simulation using three-dimensional multilayered models”, Microcirculation 2005, 12, 195-207.

The citation of any document or reference is not to be construed as an admission that it is prior art with respect to the present invention.

Thus, the present invention provides sensor systems and methods for detecting a concentration of an analyte, and more particularly provides transcutaneous oxygen tension monitor devices that provide accurate tissue pO₂ measurements without the need for long equilibration times. We have described herein the development of systems and methods which allow us to fully exploit our previously developed TCOM wearable technology, for example, with application to sensors based on O₂ quenching of phosphorescence, although these methods can be applied to transcutaneous gas sensing in general. We demonstrate experimental characterization and numerical models to extract the true tissue oxygenation. The models were tested on clinical trial data. The results show that it is possible to obtain true tissue pO₂ without waiting for long periods of time for equilibration, which currently are the main limitation to the range of application of this technology.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

What is claimed is:

1. A sensor system comprising:
a probe sensitive to an analyte, the probe having a body including a first surface and an opposite second surface;
a photon source configured to direct photons at the probe, the probe emitting light in response to receiving photons from the photon source;
a photodetector configured to detect the light emitted from the probe; and
a controller in communication with the photon source and the photodetector, the controller being configured to execute a program stored in the controller to:
(i) cause the photon source to direct photons at the probe and excite the probe to emit light in response to receiving the photons,
(ii) receive optical data from the photodetector based on the interaction between the light emitted from the probe and the photodetector,
(iii) determine a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector, and
(iv) determine a second level of the parameter of the analyte adjacent the second surface of the probe based on the difference between the first level of the parameter of the analyte and the intermediate level of the parameter of the analyte.

2. The sensor system of claim 1 wherein:
the controller executes the program stored in the controller to determine the second level of the analyte adjacent the second surface of the probe by solving a diffusion mathematical problem including the first level of the analyte and the intermediate level of the analyte.

3. The sensor system of claim 1 wherein:
the controller executes the program stored in the controller to determine the second level of the analyte adjacent the second surface of the probe by solving an inverse boundary condition mathematical problem including the first level of the analyte and the intermediate level of the analyte.

4. The sensor system of claim 3 wherein:
the inverse boundary condition mathematical problem includes a thickness dependent diffusivity variable.

5. The sensor system of claim 3 wherein:
the inverse boundary condition mathematical problem includes Dirichlet boundary conditions.

6. A sensor system comprising:
a probe sensitive to an analyte, the probe having a body including a first surface and an opposite second surface;
a photon source configured to direct photons at the probe, the probe emitting light in response to receiving photons from the photon source;
a photodetector configured to detect the light emitted from the probe; and
a controller in communication with the photon source and the photodetector, the controller being configured to execute a program stored in the controller to:
(i) cause the photon source to direct photons at the probe and excite the probe to emit light in response to receiving the photons,
(ii) receive optical data from the photodetector based on the interaction between the light emitted from the probe and the photodetector,
(iii) determine, based on the optical data from the photodetector, a gradient of a parameter of the analyte extending from a first location adjacent the first surface of the probe to an intermediate location between the first location and a second location adjacent the second surface of the probe, and
(iv) calculate, based on the gradient, a level of the parameter of the analyte adjacent the second surface of the probe.

7. The sensor system of any of claims 1-6 wherein:
the parameter is partial pressure.

8. The sensor system of any of claims 1-6 wherein:
the analyte includes at least one of oxygen, carbon dioxide, nitric oxide, and volatile organic compounds.

9. The sensor system of any of claims 1-6 wherein:
the analyte includes oxygen.

10. The sensor system of any of claims 1-6 wherein:
a section of the sensor system is adapted to define a zone adjacent a third surface when the section of the sensor system is positioned over a portion of the third surface, and
the probe is in fluid communication with the zone.

11. The sensor system of claim 10 wherein:
the third surface comprises skin of a patient, and
the analyte includes transcutaneous oxygen.

12. The sensor system of claim 10 wherein:
the probe comprises a layer of a polymeric material embedded with a metalloporphyrin.

13. The sensor system of claim 12 further comprising:
an oxygen permeable scattering layer positioned between the layer of the polymeric material and the third surface.

14. The sensor system of claim 13 wherein:
the oxygen permeable scattering layer increases collected phosphorescence signal reaching the photodetector and serves as optical insulation.

15. The sensor system of claim 12 further comprising:
a semi-permeable layer positioned between the layer of the polymeric material and the photodetector.

16. The sensor system of claim 15 wherein:
the semi-permeable layer is semi-permeable to oxygen diffusion therethrough.

17. The sensor system of claim 10 wherein:
the third surface comprises skin of a patient,
the analyte includes transcutaneous oxygen, and
an equilibration time of oxygen in the zone is less than 30 minutes when the section of the sensor system is positioned over the portion of the skin.

18. The sensor system of claim 10 wherein:
the third surface comprises skin of a patient, and
the analyte includes a liquid-dissolved compound in tissue.

19. The sensor system of claim 18 wherein:
the liquid-dissolved compound is selected from the group consisting of drugs and metabolites.

20. The sensor system of claim 10 wherein:
the third surface comprises a battery.

21. The sensor system of claim 10 wherein:
the third surface comprises a material selected from the group consisting polymeric materials, carbon-fiber materials, composite materials, and multilayered materials.

22. A method for determining a level of a parameter of an analyte, the method comprising:
(a) positioning a probe proximate to a surface to monitor an analyte;
(b) positioning a photon source to deliver photons to the probe to excite the probe;
(c) positioning a photodetector to receive light emitted by the probe in response to being emitted by the photon source;
(d) causing the photon source to direct photons at the probe and excite the probe to emit light in response to receiving the photons;
(e) collecting optical data from the photodetector based on the interaction between the light emitted from the probe and the photodetector;
(f) determining a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector; and
(g) determining a second level of the parameter of the analyte adjacent the second surface of the probe based on the difference between the first level of the parameter of the analyte and the intermediate level of the parameter of the analyte.

23. The method of claim 22 wherein:
step (g) comprises determining the second level of the analyte adjacent the second surface of the probe by solving a diffusion mathematical problem including the first level of the analyte and the intermediate level of the analyte.

24. The method of claim 22 wherein:
step (g) comprises determining the second level of the analyte adjacent the second surface of the probe by solving an inverse boundary condition mathematical problem including the first level of the analyte and the intermediate level of the analyte.

25. The method of claim 24 wherein:
the inverse boundary condition mathematical problem includes a thickness dependent diffusivity variable.

26. The method of claim 24 wherein:
the inverse boundary condition mathematical problem includes Dirichlet boundary conditions.

27. A method for determining a level of a parameter of an analyte, the method comprising:
(a) positioning a probe proximate to a surface to monitor an analyte;
(b) positioning a photon source to deliver photons to the probe to excite the probe;
(c) positioning a photodetector to receive light emitted by the probe in response to being emitted by the photon source;
(d) causing the photon source to direct photons at the probe and excite the probe to emit light in response to receiving the photons;
(e) collecting optical data from the photodetector based on the interaction between the light emitted from the probe and the photodetector;
(f) determining, based on the optical data from the photodetector, a gradient of a parameter of the analyte extending from a first location adjacent the first surface of the probe to an intermediate location between the first location and a second location adjacent the second surface of the probe; and
(g) calculating, based on the gradient, a level of the parameter of the analyte adjacent the second surface of the probe.

28. The method of any of claims 22-27 wherein:
the parameter is partial pressure.

29. The method of any of claims 22-27 wherein:
the analyte includes at least one of oxygen, carbon dioxide, nitric oxide, and volatile organic compounds.

30. The method of any of claims 22-27 wherein:
the analyte includes oxygen.

31. The method of any of claims 22-27 further comprising:
positioning a section of the sensor system over a portion of a third surface to define a zone adjacent the third surface such that the probe is in fluid communication with the zone.

32. The method of claim 31 wherein:
the third surface comprises skin of a patient, and
the analyte includes transcutaneous oxygen.

33. The method of claim 31 wherein:
the probe comprises a layer of a polymeric material embedded with a metalloporphyrin.

34. The method of claim 33 further comprising:
positioning an oxygen permeable scattering layer between the layer of the polymeric material and the third surface.

35. The method of claim 33 further comprising:
positioning a semi-permeable layer between the layer of the polymeric material and the photodetector, wherein the semi-permeable layer is semi-permeable to oxygen diffusion therethrough.

36. The method of claim 31 wherein:
the third surface comprises skin of a patient,
the analyte includes transcutaneous oxygen, and
an equilibration time of oxygen in the zone is less than 30 minutes when the section of the sensor system is positioned over the portion of the skin.

37. The method of claim 36 wherein:
the equilibration time of oxygen in the zone is less than 20 minutes.

38. The method of claim 36 wherein:
the equilibration time of oxygen in the zone is less than 10 minutes.

39. A computer system comprising instructions stored on a non-transitory computer readable medium to cause at least one processor on a computer to:
(i) cause a photon source to direct photons at a probe sensitive to an analyte and excite the probe to emit light in response to receiving the photons, the probe having a body including a first surface and an opposite second surface;
(ii) receive optical data from a photodetector based on the interaction between the light emitted from the probe and the photodetector;
(iii) determine a difference between a first level of a parameter of the analyte at a first location adjacent the first surface of the probe and an intermediate level of the parameter of the analyte at an intermediate location between the first location and a second location adjacent the second surface of the probe, wherein the intermediate level of the parameter of the analyte at the intermediate location is determined based on the optical data from the photodetector; and
(iv) determine a second level of the parameter of the analyte adjacent the second surface of the probe based on the difference between the first level of the parameter of the analyte and the intermediate level of the parameter of the analyte.

40. The computer system of claim 39 wherein:
the at least one processor determines the second level of the analyte adjacent the second surface of the probe by solving a diffusion mathematical problem including the first level of the analyte and the intermediate level of the analyte.

41. The computer system of claim 39 wherein:
the at least one processor determines the second level of the analyte adjacent the second surface of the probe by solving an inverse boundary condition mathematical problem including the first level of the analyte and the intermediate level of the analyte.

42. The computer system of claim 41 wherein:
the inverse boundary condition mathematical problem includes a thickness dependent diffusivity variable.

43. The computer system of claim 41 wherein:
the inverse boundary condition mathematical problem includes Dirichlet boundary conditions.

44. A computer system comprising instructions stored on a non-transitory computer readable medium to cause at least one processor on a computer to:
(i) cause a photon source to direct photons at a probe sensitive to an analyte and excite the probe to emit light in response to receiving the photons, the probe having a body including a first surface and an opposite second surface;
(ii) receive optical data from a photodetector based on the interaction between the light emitted from the probe and the photodetector;
(iii) determine, based on the optical data from the photodetector, a gradient of a parameter of the analyte extending from a first location adjacent the first surface of the probe to an intermediate location between the first location and a second location adjacent the second surface of the probe; and
(iv) calculate, based on the gradient, a level of the parameter of the analyte adjacent the second surface of the probe.

45. The computer system of any of claims 39-44 wherein:
the parameter is partial pressure.

46. The computer system of any of claims 39-44 wherein:
the analyte includes at least one of oxygen, carbon dioxide, nitric oxide, and volatile organic compounds.

47. The computer system of any of claims 39-44 wherein:
the analyte includes oxygen.
